(19) United States
(12) Patent Application Publication
(10) Pub. No.: US 2025/0152651 A1
(43) Pub. Date: May 15, 2025

(54) COMPOSITIONS AND METHODS FOR EXTRACTING COMPOUNDS

(71) Applicant: Unicity Properties, Inc., Provo, UT (US)

(72) Inventors: Dan Li, Singapore (SG); Liting Cai, Singapore (SG)

(21) Appl. No.: 18/943,685

(22) Filed: Nov. 11, 2024

Related U.S. Application Data
(60) Provisional application No. 63/598,277, filed on Nov. 13, 2023.

(51) Int. Cl. A61K 36/185; A61K 38/47

(52) U.S. Cl. CPC A61K 36/5777; A61K 38/47; C12Y 302/01006; C12Y 302/01015; A61K 2236/19

(57) ABSTRACT

Disclosed herein are compositions and methods for improving health and well-being in a subject, the compositions including a plant extract obtained by using an enzyme as a processing aid. Also provided herein are methods for extracting a plant extract from a plant material using an enzyme. Methods of producing hydrolyzed proteins are also disclosed.

### INCORPORATION BY REFERENCE TO ANY PRIORITY APPLICATIONS

This application claims priority to U.S. Provisional Patent Application No. 63/598,277, filed Nov. 13, 2023, which is hereby incorporated by reference in its entirety.

### FIELD

The present disclosure relates generally to compositions including compounds extracted from plant material. The present disclosure also relates to compositions for improving health and well-being in a subject, the compositions including, phytonutrient caffeine, theobromine, caffeolyquinic acids, saponins, and/or dietary fiber, the compositions formulated as a powder or a concentrated fluid. The present disclosure also relates to microencapsulation plant extract particles, and methods of making the same. The present disclosure also relates to methods for extracting a plant extract from a plant material by using an enzyme during processing, and methods of producing hydrolyzed proteins from native proteins by using enzyme hydrolysis.

### BACKGROUND

Plants provide a virtually limitless source of biologically active compounds, which play a number of important functions in the human body, including maintaining gut health, increasing cognition and attention span, boosting immunity, providing antimicrobial protection, and fighting aging. It has long been known that bioactive compounds can be extracted from plants, and the extracts used in a variety of pharmaceutical and nutraceutical products.

However, the extraction of bioactive compounds from plant matter is made difficult by the presence of cellulosic components in the plant cell walls. Traditional methods of extraction involve harsh solvents, high temperatures, and environmentally unfriendly conditions. Enzymes can be used to break down the materials within plant cell walls in order to provide better access to the bioactive compounds and make for easier, cost-effective, and more environmentally friendly extraction of the bioactive compounds from plant material.

Yerba mate, a natural source of caffeine and polyphenols with high antioxidant properties, has been used for centuries by indigenous people in South America. It is derived from the leaves of a holly plant native to the region. Yerba mate contains several beneficial compounds. Chlorogenic acids, found in plants and seeds like yerba mate, green coffee beans, and tea, support healthy brain function and promote an improved mood. Mate saponins, Unique to the yerba mate plant, support metabolic flexibility. Theobromine is Similar in structure to caffeine but found in high-quality chocolate. Theobromine promotes feelings of calm, improves focus, and reduces mental fatigue.

In some cases, methods that extract bioactive compounds from plant material using enzyme treatments can also be used to hydrolyze native proteins into hydrolyzed proteins.

### SUMMARY

The present disclosure generally relates to compositions for improving health and well-being, the compositions including a plant extract obtained by using an enzyme during processing, for example, as a processing aid, or a hydrolyzed protein obtained by enzymatic hydrolysis, and methods of making and using the compositions.

Some embodiments provided herein relate to compositions. In some embodiments, the compositions include a plant extract obtained from a plant, and a hydrolyzed protein powder. In some embodiments, the composition is formulated as a powder or concentrated fluid. In some embodiments, the plant is yerba mate or cocoa. In some embodiments, the protein powder is beef protein, whey protein, soy protein, pea protein, or rice protein. In some embodiments, the plant extract includes caffeine, theobromine, caffeoylquinic acid, rutin, or chlorogenic acid, saponin, or a combination thereof. In some embodiments, the compositions further include resistance maltodextrin. In some embodiments, the resistance maltodextrin is present in a ratio of 1 part resistance maltodextrin to 3 parts plant extract. In some embodiments, the compositions further include resistance starch. In some embodiments, the resistance starch is present in a ratio of 1 part resistance starch to 1 part hydrolyzed protein. In some embodiments, the plant extract is spray-dried with a solution including resistant maltodextrin. In some embodiments, the plant extract is spray-dried with a solution including resistant starch. In some embodiments, the plant extract is spray-dried with a solution including resistant dextrin.

Some embodiments provided herein relate to compositions for improving health and well-being in a subject. In some embodiments, the compositions include caffeine present in an amount of about 1.5% to about 5% w/w, theobromine present in an amount of at least 0.5% w/w, and total caffeoylquinic acids present in an amount ranging from about 5% to about 50% w/w. In some embodiments, the compositions are formulated as a powder or a concentrated fluid. In some embodiments, the compositions further include resistant maltodextrin (RMD). In some embodiments, the RMD is spray-dried on the powder or concentrated fluid. In some embodiments, the caffeine is present in an amount less than about 50 mg per unit dose.

Some embodiments provided herein relate to methods for extracting a plant extract from a plant material or for producing a hydrolyzed protein by using an enzyme as a processing aid. In some embodiments, the methods include providing a buffer solution, mixing the plant material or a protein with the buffer solution to form a homogenized slurry, adding an enzyme to the homogenized slurry to form a slurry-enzyme mixture, heating the slurry-enzyme mixture to a first temperature, maintaining the slurry-enzyme mixture at the first temperature, heating the slurry-enzyme mixture to a second temperature to deactivate the enzyme, maintaining the slurry-enzyme mixture at the second temperature, and separating the slurry-enzyme mixture into a solid fraction and a liquid fraction. In some embodiments, the liquid fraction includes the plant extract or the hydrolyzed protein. In some embodiments, the buffer solution includes citric acid, tricalcium citrate, and sodium hydroxide. In some embodiments, the buffer solution includes potassium dihydrogen phosphate. In some embodiments, the buffer solution includes potassium dihydrogen phosphate and dipotassium hydrogen phosphate. In some embodiments, the buffer solution has a pH of about 5.8. In some embodiments, the buffer solution has a pH between about 5.0 and about 6.2. In some embodiments, the buffer solution has a pH greater than 7. In some embodiments, the plant material or protein is in a ratio with the buffer solution in an amount of between 1:2 and 1:20. In some embodiments, the first temperature is in a range from about 40° C. to about 60° C. In some embodiments, the first temperature is maintained for a period of time ranging from about 1 hour to about 12 hours. In some embodiments, the second temperature is in a range from about 90° C. to about 99° C. In some embodiments, the second temperature is maintained for a period of time ranging from about 5 minutes to about 15 minutes. In some embodiments, the plant material includes yerba mate leaves. In some embodiments, the plant material includes cocoa pod husk. In some embodiments, the protein includes beef protein. In some embodiments, the plant extract includes caffeine, theobromine, rutin, chlorogenic acid, isomers of caffeoylquinic acids (CQA) (including, for example, 3-CQA, 4-CQA, 5-CQA, 3,4-diCQA, 3,5-diCQA, and 4,5-diCQA), polyphenols, saponin or a combination thereof. In some embodiments, the methods further include adding a second enzyme to the homogenized slurry. In some embodiments, the enzyme is Viscozyme and the second enzyme is Pectinex. In some embodiments, the enzyme is Alcalase and the second enzyme is flavourzyme. In some embodiments, the enzyme and the second enzyme are added simultaneously or sequentially. In some embodiments, the enzyme is a viscozyme, pectinex, alcalase, flavourzyme, cellulase, hemicellulase, pectinase, α-amylase, β-amylase, xylanase, β-glucanase, protease, phytase, esterase, endo-β-1,4-glucanase, cellobiohydrolase, proteinase, or a combination thereof. In some embodiments, the enzyme is added in an amount ranging from about 0.01% to about 5% w/w. In some embodiments, the methods further include spray-drying the plant extract with a solution including resistant maltodextrin (RMD).

Some embodiments provided herein relate to methods for improving health or well-being in a subject. In some embodiments, the methods include ingesting any of the compositions described herein. In some embodiments, the compositions include a plant extract obtained from a plant, and dietary fiber. In some embodiments, the compositions include a hydrolyzed protein and dietary fiber. In some embodiments, improving health or well-being includes improving blood lipid levels, reducing body weight, reducing fasting blood sugar levels, increasing feelings of positive experiences, or decreasing feelings of negative experiences. In some embodiments, the compositions include caffeine present in an amount of about 1.5% to about 5% w/w, theobromine present in an amount of about 0.5% to about 1% w/w, rutin present in an amount of about 0.5% to about 2% w/w, and total caffeoylquinic acids present in an amount ranging from about 5% to about 50% w/w. In some embodiments, the composition is formulated as a powder or concentrated fluid. In some embodiments, the plant is yerba mate or cocoa pod husk. In some embodiments, the protein powder is beef protein, whey protein, soy protein, pea protein, or rice protein. In some embodiments, the plant extract includes caffeine, theobromine, caffeoylquinic acid, rutin, or chlorogenic acid, or a combination thereof. In some embodiments, the composition further includes resistance maltodextrin. In some embodiments, the resistance maltodextrin is present in a ratio of 1 part resistance maltodextrin to 3 parts plant extract. In some embodiments, the composition further includes resistance starch. In some embodiments, the resistance starch is present in a ratio of 1 part resistance starch to 1 part hydrolyzed protein. In some embodiments, the plant extract is spray-dried with a solution including resistant maltodextrin. In some embodiments, the plant extract is spray-dried with a solution including resistant starch. In some embodiments, the plant extract is spray-dried with a solution including resistant dextrin.

Further disclosed herein are methods of extracting a plant extract from plant material. In some embodiments, the method includes providing plant material; treating said plant material with one or more enzymes comprising a pectinase, a hemicellulase, a beta-glucanase, or a xylanase; and extracting the plant extract from the treated plant material, wherein the extraction is conducted at a pH of 5-6.

In some embodiments, the plant material is yerba mate. In some embodiments, the plant extract is a saponin. In some embodiments, the saponin is matesaponin 1, matesaponin 2, matesaponin 3, matesaponin 4, matesaponin 5, mate saponin A, mate saponin B, ursolic acid and derivatives thereof, or oleanolic acid or derivatives thereof.

In some embodiments, the one or more enzymes include a first blend. In some embodiments, the first blend includes pectinase, hemicellulase, and beta-glucanase. In some embodiments, the one or more enzymes include a second blend. In some embodiments, the second blend includes beta-glucanase, pectinase, hemicellulase, and xylanase. In some embodiments, the first blend comprises Pectinex® and the second blend comprises Viscozyme®. In some embodiments, the first blend and second blend are used in a ratio of about 1:1.

In some embodiments, the method further includes breaking down yerba mate cell walls and releasing saponins. In some embodiments, the one or more enzymes are used at a concentration of about 0.5%. In some embodiments, the extraction yields a saponin content of at least 10%. In some embodiments, the extracted saponins exhibit antioxidant activity as measured by DPPH.

### BRIEF DESCRIPTION OF THE DRAWINGS

The foregoing and other features of the present disclosure will become more fully apparent from the following description, taken in conjunction with the accompanying drawings. Understanding that these drawings depict only some embodiments in accordance with the disclosure and are therefore, not to be considered limiting of its scope, the disclosure will be described with additional specificity and detail through use of the accompanying drawings.

FIG. 1 provides a flowchart of a method of extracting an extract from yerba mate leaf cuts according to some embodiments of the present disclosure.

FIG. 2 provides a typical High Performance Liquid Chromatogram (HPLC) unique to yerba mate extracts.

FIG. 3 provides a flowchart of a method of quantifying saponins from yerba mate extract used in Example 7.

FIG. 4 is a graph showing α-glucosidase inhibition of Yerba mate extract using different extraction conditions.

### DETAILED DESCRIPTION

The present disclosure can be understood more readily by referencing the following detailed description, examples, drawings, and claims, and their previous and following descriptions. However, before the present compositions and methods are disclosed and described, it is to be understood that this disclosure is not limited to the specific compositions and methods disclosed unless otherwise specified. It is also to be understood that the terminology used herein is for the purpose of describing particular aspects only and is not necessarily intended to be limiting.

The description is provided as an enabling teaching of the disclosure. To this end, those skilled in the relevant art will recognize and appreciate that many changes can be made to the various aspects of the disclosure described herein while still obtaining beneficial results. It will also be apparent that some of the desired benefits can be obtained by selecting some of the features described herein without utilizing other features. Accordingly, those who work in the art will recognize that many modifications and adaptations to the present description are possible and can even be desirable in certain circumstances and are a part of the present disclosure. Thus, this description is provided as illustrative of certain principles of the present disclosure and not in limitation thereof.

As summarized herein, aspects of the compositions and methods for improving health and well-being using a composition that includes an enzyme and a plant extract are provided herein.

It is to be understood that this disclosure is not limited to particular embodiments described, as such may, of course, vary. It is also to be understood that the terminology used herein is for the purpose of describing particular embodiments only, and is not intended to be limiting.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definitions

Unless defined otherwise, technical and scientific terms used herein have the same meaning as commonly understood by one of ordinary skill in the art to which the present disclosure belongs. For purposes of the present disclosure, the following terms are defined below.

All publications, patent applications, patents, and other references mentioned herein are incorporated by reference in their entirety. In case of conflict, the present specification, including explanations of terms, will control. In addition, the materials, methods, and examples are illustrative only and not intended to be limiting.

In some embodiments, provided herein are methods, kits, and compositions for improving health and well-being using a composition that includes an enzyme and a plant extract.

Advantageously, the methods, kits, and compositions described herein include improved methods for extracting plant extracts from a plant material by improving extraction yield, decreasing production costs, and decreasing processing times. The methods and compositions described herein have several advantages over conventional extraction methods, such as the use of mild environmental conditions, high bioactive yield, reduced amounts of undesirable products, and lower environmental impacts. The extraction method with enzymes results in a composition that includes bioactives and other nutrients such as protein and fiber, which is used to provide improved health and well-being to a user.

As used herein, the term “about” or “approximately” in reference to a numerical value for a particular parameter is inclusive of the numerical value and a degree of variance from the numerical value that one of ordinary skill in the art would understand is within acceptable tolerances for the particular parameter. For example, “about” or “approximately” in reference to a numerical value may include additional numerical values within a range of from 80.0 percent to 120.0 percent of the numerical value, such as within a range of from 90.0 percent to 110.0 percent of the numerical value, within a range of from 95.0 percent to 105.0 percent of the numerical value, within a range of from 97.5 percent to 102.5 percent of the numerical value, within a range of from 99.0 percent to 101.0 percent of the numerical value, within a range of from 99.5 percent to 100.5 percent of the numerical value, or within a range of from 99.9 percent to 100.1 percent of the numerical value.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those of skill within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

Any of the features of an embodiment of the first through second aspects is applicable to all aspects and embodiments identified herein. Moreover, any of the features of an embodiment of the first through third aspects is independently combinable, partly or wholly with other embodiments described herein in any way, e.g., one, two, or three or more embodiments may be combinable in whole or in part. Further, any of the features of an embodiment of the first through third aspects may be made optional to other aspects or embodiments.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, the term “composition” has its ordinary meaning as commonly understood by those skilled in the art, and includes a material or compound composed of two or more different substances, or a product containing two or more substances, whether resulting from chemical union or from mechanical mixture of the substances, and whether the substances are gases, fluids, powders or solids.

As used herein, the term “plant extract” has its ordinary meaning as commonly understood by those skilled in the art, and includes a process of removing material, compounds, compositions, or other components from a starting plant material. A process of extraction can be performed by various means, including by physical or chemical extraction, such as by pressing, grinding, heating, stirring, or other known methods for extracting a component from a starting material.

As used herein, the term “obtained” has its ordinary meaning as commonly understood by those skilled in the art, and includes originating from, being generated from, and being synthesized from a designated source(s) by known purification, separation, and/or extraction techniques.

As used herein, the term “combination thereof” has its ordinary meaning as commonly understood by those skilled in the art and includes a mixture, a blend, a stack, a reaction product, or any combination, whether singular or plural, of two or more constituting elements.

As used herein, the term “plant” or “plant material” has its ordinary meaning as commonly understood by those skilled in the art, and includes a whole plant or any parts or derivatives thereof, such as plant cells, plant protoplasts, plant cell tissue cultures from which plants can be regenerated, plant calli, embryos, pollen, ovules, fruit, flowers, leaves, seeds, roots, root tips, and the like. The term “botanical” is used herein interchangeably with plant, and refers to a plant or any parts derived thereof.

### Plant Extracts

In some embodiments, the plant is any plant having components therein that are desirable to extract, purify, or obtain from the plant. For example, the plant may be, or may be derived from, maca, he shou wu, iporuru (Alchornea castaneifolia), kanna (Sceletium tortosum), honokiol (Magnolia grandiflora), jujube (Ziziphi Spinosae), cnidium (Fructus cnidii), corydalis (Corydalis yanhusuo), albizia (Cortex albiziae), ginseng (Panax ginseng), polygonum (Polygoni multiflori), fu ling (Poria cocos), cornus (Fructus corni), Chinese yam (Rhizoma dioscoreae), muira puama, Dendrobium sp., licorice root radix (Glycyrrhizae preparata), cordyceps (Cordyceps sinensis), Chinese angelica (Angelicae sinensis), kratom (Mitragyna speciosa), bacopa monnieri, catuaba, ashwaghanda, peganum harmala, wheat, alfalfa, oat, kamut, echinacea, chlorella, amla, stinging nettles, carob, mesquite, chuchuhuasai, clavo huasca, chanca piedra, guayusa, Rhodiola rosea, shilajit, higenamine, moringa (Moringa oleifera), horny goat weed (Epidmedium), astragalus, aloe vera, turmeric, pine, curcumin (turmeric compound), hops, xanthohumol (hops compound), passion flower, mucuna puriens, tusli, black pepper, bioperine (black pepper compound), Siberian ginseng, American ginseng, yerba mate, lemon balm, astragulus, kava, schizandra, skullcap, valerian, California poppy, epidmedium, pau d′arco, gingko, lotus, lily, tea, coffee, or cacao, or any components of such plants, including bark, leaves, stems, roots, flowers, fruits, pollens, seeds or the like. Plant ingredients may include plant oils, including, for example linalool; b-caryophyllene; b-myrcene; d-limonene; humulene; α-pinene; ylang (Cananga odorata); yarrow (Achillea millefolium); violet (Viola odorata); vetiver (Vetiveria zizanoides); vanilla (Vanilla plantifolia); tuberose (Polianthes tuberosa); thyme (Thymus vulgaris L.); tea tree (Melaleuca alternifolia); tangerine (Citrus reticulata); spruce (Picea mariana); spruce (Tsuga canadensis); zpikenard (Nardostachys jatamansi); spearmint (Mentha spicata); sandalwood (Santalum spicatum); rosewood (Aniba rosaeodora); rosemary verbenone (Rosmarinus officinalis); rosemary (Rosmarinus officinalis); rose (Rosa damascena); rose geranium (Pelargonium roseum); ravensara (Ravensara aromatica); plai (Zingiber cassumunar); pine needle (Pinus sylvestris L.); petitgrain (Citrus aurantium); peppermint (Mentha piperita); pepper (Piper nigrum L.); patchouli (Pogostemon cablin); palo santo (Bursera graveolens); palmarosa (Cymbopogon martini); osmanthus (Osmanthus fragrans); oregano (Origanum vulgare); orange (Citrus sinensis); oak moss (Evernia prunastri); nutmeg (Myristica fragrans); niaouli (Melaleuca viridifloria); neroli (Citrus aurantium); myrtle (Myrtus communis); myrrh (Commiphora myrrha); mimosa (Acacia decurrens); melissa (Melissa officinalis L.); marjoram (Origanum majorana); manuka (Leptospermum scoparium); mandarin (Citrus deliciosa); lotus (Nelumbo nucifera); lime (Citrus aurantifolia); lily (Lilum aurantum); lemongrass (Cymbopogon citratus); lemon (Citrus limonum); lavender (Lavandula angustifolium); lavandin (Lavandula hybrida grosso); kanuka (Kunzea ericoides); juniper (Juniperus cummunis); jasmine (Jasminum officinale); jasmine (Jasminum sambac); helichrysum (Helichrysum italicum); grapefruit (Citrus xparadisi); grapefruit (Citrus paradisi); ginger (Zingiber officinalis); geranium (Pelargonium graveolens); geranium (Pelargonium graveolens, ‘Herit); gardenia (Gardenia jasminoides); galbanum (Ferula galbaniflua); frankincense (Boswellia carterii); frangipani (Plumeria alba); fir needle white (Abies alba); fir needle Siberia (Abies siberica); fir needle Canada (Abies balsamea); fennel (Foeniculum vulgare); Eucalyptus smithii. Eucalyptus radiata, Eucalyptus globulus, Eucalyptus citriodora, Eucalyptus Blue Mallee (Eucalyptus polybractea); elemi (Canarium luzonicum); dill (Anethum graveolens); cypress (Cupressus sempervirens); cumin (Cuminum cyminum); coriander (Coriandum sativum); cocoa (Theobroma cacao); clove (Eugenia caryophylatta); clary sage (Salvia sclarea); cistus (Labdanum) (Cistus ladaniferus L.); cinnamon (Cinnamomum zeylanicum); chamomile (Anthemis nobilis); chamomile (Matricaria chamomilla); celery seed (Apium graveolins); cedarwood (Thuja plicata); cedarwood (Juniperus virginiana); cedarwood atlas (Cedrus atlantica); carrot seed (Daucus carota); cardamon (Elettaria cardamomum); caraway seed (Carum carvi); cajeput (Melaleuca cajuputi); cade (Juniperus oxycedrus); birch (Betula alba); birch (Betula lenta); bergamot (Citrus bergamia); bay laurel (Laurus nobilis); basil (Ocimum basilicum); basil (Ocimum sanctum); basil (Ocimum basilicum); balsam poplar (Populus balsamifera); balsam Peru (Myroxylon balsamum); or angelica (Angelica archangelica L.).

As used herein, the term “bioactive compound” has its ordinary meaning as commonly understood by those skilled in the art, and includes any chemical or biological compound(s) derived from a plant and that causes a specific effect on a living organism upon exposure of the living organism to the bioactive compound. Non-limiting examples of effects caused by bioactive compounds include anti-inflammatory effects, antioxidant effects, antimicrobial and/or antiviral effects, anti-cancer, anti-stress, anti-aging, and/or anti-neurodegenerative effects.

As used herein, the term “enzyme” has its ordinary meaning as understood in light of the specification, and refers to any protein capable of producing changes in a biological substance by catalytic action.

As used herein, the term “cellulase” has its ordinary meaning as commonly understood by those skilled in the art, and includes a diverse group of enzymes that catalyze the decomposition of cellulose and/or related polysaccharides. In particular, cellulases break down the cellulose molecule into monosaccharides or shorter polysaccharides by hydrolyzing the chain ends binding adjacent sugar molecules together.

As used herein, the term “hemicellulase” has its ordinary meaning as commonly understood by those skilled in the art, and includes a diverse group of enzymes that hydrolyze hemicellulose polysaccharides including xylans, xyloglucans, arabinoxylans, and glucomannans, into monosaccharides or shorter polysaccharides by hydrolyzing the chain ends binding adjacent sugar molecules together.

As used herein, the term “pectinase” has its ordinary meaning as commonly understood by those skilled in the art, and includes a diverse group of enzymes that break down pectin through hydrolysis, transelimination, and/or deesterification reactions. Non-limiting examples of pectinases include pectolyase, pectozyme, and polygalacturonase.

In some embodiments, the methods and compositions described herein include extraction of yerba mate. As used herein, the term “yerba mate” has its ordinary meaning as commonly understood by those skilled in the art, and refers to the medicinal plant, Ilex paraguariensis.

Yerba mate, dried leaves, and stemlets of the perennial tree Ilex paraguariensis St. Hilaire (Luxner, 1995) are commonly used to prepare infusions widely consumed in South America. Yerba mate has a rich bioactive content. The tea like infusion was claimed by its depurative, diuretic, and stimulant properties. It was reported that yerba mate helps to increase the antioxidant defense of the organism against free radicals. Yerba mate also displays an antiglycation action, which contributes to avoid diabetic chronic complications. Consumption of yerba mate infusions is important for nutritional purposes since yerba mate is a rich source of minerals, such as K, Mg, and particularly the trace element Mn. In addition to minerals, yerba mate infusions contain xanthines such as caffeine, theobromine, and theophylline, saponins, and caffeoylquinic acids, a family of mono- and di-acyl quinic acids.

Yerba mate contains various important compounds. It is a natural source of caffeine, and is a mild central nervous system stimulant. Yerba mate has a moderate caffeine content (1-2% by dry weight) compared to coffee (1-2.5%), but more than most teas, offering alertness and energy (Burris et al., 2012) (Schmalko and Alzamora, 2001; Bastos et al., 2005; Calviño, Tamasi, and Ciappini, 2005). In addition to caffeine, yerba mate also contains other xanthines, such as theobromine and theophylline that have stimulant effects akin to caffeine (Filip et al., 1998). Theobromine is found in chocolate (Martínez-Pinilla, Oñatibia-Astibia and Franco, 2015), while theophylline is used in bronchodilator medications (Weinberger et al., 1975). Rich in polyphenolic compounds, including flavonoids and phenolic acids, yerba mate provides antioxidants that may shield cells from oxidative damage (Bastos et al., 2007; C.I. Heck and Mejia, 2007; C. I. Heck and De Mejia, 2007; Burris et al., 2012; Gullón et al., 2018; Gan et al., 2018; Gómez-Juaristi et al., 2018; Schuster and Mitchell, 2019; Gawron-Gzella, Chanaj-Kaczmarek, and Cielecka-Piontek, 2021; Rząsa-Duran et al., 2022; de Vasconcellos, Frazzon, and Zapata Noreña, 2022; Burris et al., no date). Chlorogenic acid, a type of polyphenol, is also found in coffee and has associated health benefits. Caffeoyl derivatives contribute to the unique flavor and aroma of yerba mate, possibly having antioxidant properties (Martinet, Hostettmann, and Schutz, 1999; Puangpraphant et al., 2011; da Silveira et al., 2016). Yerba mate contains saponins, natural compounds known for potential health benefits, including anti-inflammatory and cholesterol-lowering properties (Ferreira et al., 1997; Puangpraphant and De Mejia, 2009; Petroselli et al., 2019).

Yerba mate includes various vitamins (A, C, and B-complex vitamins like B1, B2, B3, and B5) and essential minerals such as potassium, magnesium, and manganese. Several essential amino acids, including arginine, leucine, and lysine, are present, playing crucial roles in metabolic processes (Bragança, Melnikov, and Zanoni, 2011; Conforti, Gallo, and Saraví, 2012; Bastos et al., 2014).

Typically, a 150 mL cup of yerba mate tea contains 10-78 mg of caffeine (Heck & Mejia, 2007), and the level can vary depending on the amount of tea, the volume of boiled water used, the brewing temperature and the length of brewing time. In the human body, the low level of caffeine achieved by drinking such beverages mostly works as a mild central nervous stimulant. Caffeine is a natural chemical with stimulant effects. It is found in coffee, tea, cola, cocoa, guarana, yerba mate, and over 60 other products (Heckman et al., 2010). Studies have linked caffeine consumption with various human health benefits such as enhancement of cognitive functioning (Foskett et al., 2009; Lorist & Tops, 2003), improvement of neuromuscular coordination (Glade, 2010; Samoggia & Rezzaghi, 2021; San Juan et al., 2019), elevation of mood (Herz, 1999; Lieberman et al., 1987), relief of anxiety (Greden, 1974; Smith, 1988), and stimulation of the central nervous system and the cardiac muscle (Bolton & Null, 1981; Davis et al., 2003). Therefore, caffeine has been used as an additive for soft drinks and energy drinks. Caffeine has also been added to pharmaceuticals to improve analgesic effects (Derry et al., 2014; Ward et al., 1991; W.-Y. Zhang, 2001).

However, caffeine also has a few disadvantages. This includes a bitter taste, and development of caffeine dependence which may lead to anxiety or increased jitteriness. Specifically, low-caffeine plant extract products, including yerba mate, can offer several potential benefits. Lower caffeine levels can help individuals avoid the jittery or anxious feelings that can accompany higher caffeine intake. Lower caffeine levels can improve sleep, and can help individuals stay focused without experiencing increased caffeine-induced restlessness. Lower caffeine levels may also provide a milder and more sustained energy boost, while being gentler on the digestive system. For these reasons, plant extracts having lower caffeine levels, while at the same time retaining high levels of polyphenols, antioxidants, and other bioactive compounds, are needed.

Cocoa pod husk is the external portion of the cocoa pod. The cocoa pod consists of the husk, the pulp, and the cocoa beans. The husk constitutes the majority of the whole pod: about 52-76% of the cocoa pod. Donkoh et al., Anim Feed Sci Technol. 1991, 35, 161-169. Nevertheless, the husk is typically considered a waste product by the cocoa industry and is usually discarded, used as a natural fertilizer, or included in animal feed. However, cocoa pod husks contain a wide variety of bioactive compounds, including fiber, minerals, theobromine, and polyphenols, many of which are expected to have substantial health benefits. The potential valorization of cocoa shell waste offers several benefits in terms of sustainability, economics, environmental impact, nutrition, and energy generation, making it a promising new avenue for the cocoa industry.

Bioactive compounds in the plant matter, including polyphenols, are interspersed within this complex matrix and also bound to it via strong noncovalent interactions. The extraction of bioactive compounds from the matrix requires detaching them from the multitude of physical and chemical interactions with the matrix components, so as to release the bioactive compounds while leaving the unwanted inert and fibrous components behind.

Generally, the extraction of bioactive compounds from plant matter is made difficult by the presence of cellulosic components in the plant cell walls. The plant cell wall is a complex structure serving as both a protective barrier and a vital component in plant development and growth. It consists of a matrix of carbohydrates and glycoproteins with primary constituents including lignin, cellulose, hemicellulose, and pectins. Lignin provides structural support to the cell wall, while cellulose forms a rigid framework, composed of long chains of glucose molecules, Hemicelluloses, like xyloglucan, are interspersed among the cellulose fibers, adding to the complexity. Pectins, including polygalacturonic acid and rhamnogalacturonan, form a matrix around the cellulose-hemicellulose network. There remains a need for improved methods for extracting bioactive compounds from plant materials, in order to increase the efficiency of extracting the bioactive compounds from the plant material, particularly the plant cell walls, while maintaining their antioxidant properties.

Traditional methods of extracting compounds from plant matter typically utilize harsh solvents such as aqueous mixtures of ethyl alcohol, combined with heating and physical mixing to separate the bioactive compounds from the fibrous components of the plant cell wall. Craft et al., Compreh. Rev. Food Si. and Food Safety, 2012, 11, 148-173. However, solvent-based extraction methods generally have low efficiency, high environmental impact, and high energy costs.

To overcome some of the deficiencies of traditional methods of extracting bioactive compounds from plant matter, methods including the use of enzyme pre-treatments have been studied. Enzymes, which are proteins that catalyze chemical reactions, have been employed to break down the materials within plant cell walls in order to provide better access to the bioactive compounds contained therein. For example, Heemann et al. described enzyme-assisted extraction of bioactive compounds from yerba mate using a commercial blend of carbohydrase enzymes. Heemann et al., Braz. J. Food Technol. 2019, 22, e2017222, 1-10. The authors utilized a chemical assay, the Folin Ciocalteau reagent, which is based on the chemical reduction of a molybdenum- and tungsten-containing reagent which reacts with polyphenols, to measure the total extracted polyphenol content. They found that the use of carbohydrases increased the extraction of polyphenols from about 39% to about 52%.

Similarly, Krakowska-Sieprawska et al. tested enzyme extraction as a pretreatment to supercritical fluid extraction (SFE) of yerba mate leaves, using a commercial enzyme blend, Kemzyme™. Krakowska-Sieprawska 2020. They found that enzyme pretreatment in SFE achieved a final level of total phenolic compounds approximately 3.2 higher than SFE without enzyme extraction pretreatment.

Similar approaches that can be used for extracting bioactive compounds from plant material can sometimes be used to produce hydrolyzed proteins from native proteins. Native proteins, which are proteins in their naturally occurring or unmodified form, can have several limitations in various applications. Some native proteins can be challenging to digest, leading to gastrointestinal discomfort for some individuals, especially those with sensitivities or allergies to specific proteins. Certain native proteins are common allergens, such as those found in milk, eggs, soy, and peanuts. Allergies to these proteins can be severe and life-threatening. Many native proteins are sensitive to heat and can denature or coagulate at elevated temperatures. This can affect the texture and functionality of food products during cooking or processing. Native proteins may not provide the desired texture or stability in food products. For example, they can lead to separation or phase changes in some formulations. Some native proteins have strong or off-putting flavors that can be undesirable in certain food applications. In some cases, native proteins can bind to essential nutrients, making them less bioavailable for absorption in the digestive system. Native proteins can be prone to spoilage and microbial contamination, reducing the shelf life of products. Extracting and using native proteins can be expensive due to factors like production, purification, and transportation costs.

To address these challenges, various food and biotechnology industries often employ protein modification techniques, such as hydrolysis, denaturation, or genetic engineering, to improve protein functionality, reduce allergenicity, enhance digestibility, and tailor proteins for specific applications. Several of these methods utilize harsh solvents and/or heat that disrupt the taste, texture, or other properties of the product containing the protein. Genetic engineering comes with significant complexity and expense. Therefore, improved methods of hydrolyzing native proteins to hydrolyzed proteins are needed that operate under mild conditions, without the harsh chemicals, high environmental impact, and high cost of previous methods.

Different enzymes can be used for the extraction of bioactive compounds from plant material and/or for the hydrolysis of native proteins to hydrolyzed proteins (Puri, Sharma and Barrow, 2012; Nadar, Pawar, and Rathod, 2017; Nadar, Rao, and Rathod, 2018). Cellulase is an enzyme that breaks down cellulose, a major component of plant cell walls (Levy, Shani, and Shoseyov, 2002; Doi and Kosugi, 2004; Wilson, 2008; Martínez et al., 2009). It is used to extract a variety of compounds from plant materials, including sugars, proteins, and vitamins. Pectinase is an enzyme that breaks down pectin, another major component of plant cell walls. It is used to extract juice from fruits and vegetables, and to clarify wine and beer (Ali et al., 2005; Pedrolli et al., 2009; Pakarinen et al., 2012; Pasha, Anuradha and Subbarao, 2013; Tapre and Jain, 2014; Shet, Desai, and Achappa, 2018). Amylase is an enzyme that breaks down starch (Macfarlane and Englyst, 1986; Bosch et al., 2011; Butterworth, Warren, and Ellis, 2011; Jain et al., 2020), a type of carbohydrate. It is used to make bread, beer, and other food products. Lipase is an enzyme that breaks down lipids, or fats (Gandhi, 1997; Houde, Kademi, and Leblanc, 2004; Pirahanchi and Sharma, 2019). It is used to extract oils from plants and animals, and to make soaps and detergents. Protease is an enzyme that breaks down proteins (Stroud, 1974; Barrett, 2000). It is used to make cheese, yogurt, and other dairy products, and to tenderize meat. Xylanase is an enzyme that breaks down xylan (Grüninger and Fiechter, 1986; Liab et al., 2000; Dodd and Cann, 2009), a type of carbohydrate found in plant cell walls. It is used to extract sugars from plants, and to make biofuels. These are just a few of the many enzymes that can be used for hydrolysis. The specific enzyme that is used will depend on the compound that is being targeted for extraction and the desired application.

When choosing an enzyme for a particular application, it is important to select one that is specific for the desired reaction because Enzymes are highly specific, meaning that they will only catalyze a particular reaction or a small group of related reactions. This specificity is due to the unique three-dimensional structure of the enzyme, which creates a binding site that is complementary to the shape of the substrate molecule.

The concentration of the enzyme will also affect the efficiency of the extraction process. Generally, higher enzyme concentrations will result in higher yields of the target compound. However, it is important to avoid using too high a concentration of enzyme, as this can lead to inhibition of the enzyme. The substrate concentration is the amount of the target compound that is present in the starting material. The higher the substrate concentration, the more efficient the extraction process will be. However, it is important to avoid using too high a substrate concentration, as this can also lead to inhibition of the enzyme. The temperature of the extraction process will also affect the efficiency of the extraction process. Generally, higher temperatures will result in faster extraction rates. However, it is important to avoid using temperatures that are too high, as this can denature the enzyme and reduce its effectiveness. The pH of the extraction process will also affect the efficiency of the extraction process. Generally, enzymes have an optimum pH at which they are most effective. Using a pH that is too high or too low can reduce the effectiveness of the enzyme. The time of the extraction process will also affect the efficiency of the extraction process. Generally, longer extraction times will result in higher yields of the target compound. However, it is important to avoid extracting for too long, as this can also lead to degradation of the target compound. The substrate may need to be pretreated before enzyme hydrolysis to improve the efficiency of the extraction process. Pretreatment methods can include physical methods, such as grinding or milling, or chemical methods, such as solvent extraction or acid hydrolysis.

Yerba mate (Ilex paraguariensis) contains a significant amount of saponins, which are bitter, water-soluble triterpene compounds derived from ursolic acid. (Gawron-Gzella et al., Nutrients, 2021 Oct. 21; 13 (11): 3706. doi: 10.3390/nu13113706). The primary saponins identified in yerba mate are matesaponins 1 through 5, with matesaponins 1 and 2 being the most abundant. (Mateos et al., J. Food Composition and Analysis, Vol. 63, 2017, 164-170). These saponins comprise approximately 5-10% of the total dry weight of yerba mate leaves. The chemical structure of matesaponin 1 is ursolic acid-3-0-[β-D-glucopyranosyl-(1→3)-α-L-rhamnopyranosyl-(1→2)-α-L-arabinopyranoside]. Yerba mate saponins have demonstrated various health benefits, including anti-inflammatory, antioxidant, and anti-obesity effects. (Gambero & Ribeiro, Nutrients, 2015 Jan. 22; 7 (2): 730-750. doi: 10.3390/nu7020730). In vitro and in vivo studies have shown that these saponins can inhibit inflammatory markers such as iNOS and COX-2 in macrophages through NFκB pathways, especially when combined with other compounds like quercetin. (Jannus et al., Int J Mol Sci. 2021 Jul. 29; 22 (15): 8158. doi: 10.3390/ijms22158158). Furthermore, yerba mate saponins have exhibited potential in reducing fat weight, plasma triglyceride levels, and both liver and fat accumulation in animal models. (Kim et al., BMC Complement Altern Med. 2015 Sep. 25; 15:338. doi: 10.1186/s12906-015-0859-1). These compounds may also contribute to the overall antioxidant activity of yerba mate, which has been reported to be similar to that of green tea. The unique composition and bioactivity of yerba mate saponins make them promising candidates for developing novel therapeutic agents targeting inflammation, obesity, and related metabolic disorders. (Alkhatib & Atcheson, Nutrients 2017, 9 (8), 882; doi.org/10.3390/nu9080882).

Previous research on yerba mate explored methods to enhance the recovery of bioactives and antioxidant properties using enzyme hydrolysis and extraction. Key factors influencing enzyme activity included enzyme-to-substrate ratio, buffer amount, temperature, time, and pH.

The use of herbal remedies as complementary treatments for diabetes is growing worldwide. Traditional literature identifies over 800 plants with antidiabetic properties, while ethnopharmacological surveys suggest more than 1,200 plants can exhibit hypoglycemic activity. Two key enzymes, α-amylase and α-glucosidase, are responsible for postprandial hyperglycemia (high blood sugar after meals). α-Amylase breaks down complex carbohydrates like starch and glycogen into disaccharides, while α-glucosidase further converts these disaccharides into monosaccharides (simple sugars), leading to increased blood glucose levels. Inhibiting these enzymes can delay carbohydrate digestion and help control postprandial glucose spikes, making such inhibitors useful in managing hyperglycemia.

Yerba mate has shown promising antidiabetic effects due to its rich chemical composition, which includes polyphenols (such as chlorogenic acid), xanthines (caffeine and theobromine), and saponins. These compounds contribute to several mechanisms that positively influence glucose metabolism and insulin sensitivity, both crucial for diabetes management.

### Mechanisms of Action

Yerba mate has shown promising antidiabetic effects due to its rich chemical composition, which includes polyphenols (such as chlorogenic acid), xanthines (caffeine and theobromine), and saponins. These compounds contribute to several mechanisms that positively influence glucose metabolism and insulin sensitivity, both crucial for diabetes management.

Improved Insulin Sensitivity and Secretion: Yerba mate enhances insulin sensitivity and stimulates insulin secretion. Studies have shown that yerba mate increases glucose-stimulated insulin secretion (GSIS) in pancreatic islets, which is essential for maintaining glucose balance. In animal models, yerba mate consumption elevated mRNA levels of key proteins in the insulin signaling pathway, such as IRS-1 and PI3K, while reducing inflammatory markers like TNF-α. This indicates that yerba mate not only improves insulin sensitivity but also reduces inflammation in pancreatic cells, aiding in better glucose control (Maiztegui et al., Plants (Basel), 2023 Jul. 12; 12 (14): 2620. doi: 10.3390/plants12142620).

Inhibition of SGLT-1: Yerba mate has been shown to inhibit the expression of sodium-glucose transporter-1 (SGLT-1) in the intestines of diabetic rats. SGLT-1 facilitates glucose absorption from the gut into the bloodstream. By reducing SGLT-1 activity, yerba mate may help lower postprandial glucose levels, contributing to better glycemic control.

Antioxidant Properties. The polyphenols in yerba mate, particularly chlorogenic acid and caffeic acid, exhibit strong antioxidant properties that help reduce oxidative stress, a key factor in diabetes progression. Oxidative stress can impair insulin signaling and worsen insulin resistance. By scavenging free radicals and reducing lipid peroxidation, yerba mate may protect pancreatic beta cells from damage and improve their function (Gawron-Gzella et al., Nutrients. 2021 Oct. 21; 13 (11): 3706. doi: 10.3390/nu13113706).

Reduction of Advanced Glycation End-products (AGEs): AGEs are harmful compounds formed when proteins or fats combine with sugars in the bloodstream, contributing to diabetes complications such as neuropathy and retinopathy. Yerba mate has been shown to reduce AGE formation, potentially mitigating some long-term complications associated with diabetes.

Lipid Metabolism Improvement: In addition to its effects on glucose metabolism, yerba mate positively influences lipid metabolism by reducing cholesterol levels and improving lipid profiles. This is particularly beneficial for individuals with type 2 diabetes (T2D), who often suffer from dyslipidemia (abnormal lipid levels). Yerba mate helps lower LDL cholesterol and triglycerides while enhancing fat oxidation, further supporting metabolic health in diabetic patients (Rocha et al., Biomedicine & Pharmacotherapy, vol. 105, September 2018, Pages 370-376).

### Human Studies:

Human trials have also supported yerba mate's antidiabetic potential. In a randomized controlled trial, regular consumption of yerba mate significantly reduced fasting blood glucose and improved insulin levels in participants with pre-diabetes or T2D. Long-term supplementation with yerba mate was associated with reductions in hemoglobin A1c (HbA1c), a marker of long-term blood sugar control56. Additionally, it improved lipid profiles by lowering LDL cholesterol and triglycerides while enhancing antioxidant capacity (Sarria et al, Proceedings of the Nutrition Society. 2020; 79 (OCE2): E245. doi: 10.1017/S0029665120001937).

Yerba mate shows significant potential as a complementary therapy for managing diabetes due to its multiple mechanisms of action-enhancing insulin sensitivity, reducing postprandial glucose absorption, protecting pancreatic cells from oxidative damage, lowering AGEs formation, and improving lipid metabolism. These effects make it a promising candidate for further research and potential integration into diabetes management strategies alongside conventional treatments.

### Compositions

Some embodiments herein relate to compositions that include a plant extract obtained from a plant or a hydrolyzed protein powder. In some embodiments, the composition is formulated as a powder or concentrated fluid.

In some embodiments, the composition includes a plant extract that is extracted from a plant using any of the methods described herein, including processing a plant material with an enzyme. In some embodiments, the enzyme is a carbohydrase. In some embodiments, the enzyme includes cellulase, hemicellulase, pectinase, α-amylase, β-amylase, xylanase, β-glucanase, protease, phytase, esterase, endo-β-1,4-glucanase, cellobiohydrolase, proteinase, or a combination thereof. In some embodiments, the enzyme is an endo-β-glucanase or a polygalacturonase.

In some embodiments, a combination of enzymes may be provided. For example, the combination of enzymes may be provided in a commercial enzyme product, such as Kemzymes™, Viscozyme™, Pectinex™ or any other mixture.

Viscozyme and Pectinex are multi-enzyme complexes encompassing a broad spectrum of carbohydrate-hydrolyzing enzymes. These complexes find utility in extracting various components from plant materials. Viscozyme, in particular, acts on non-starch polysaccharides and pectin-like substances in plant cell walls. By doing so, it reduces the viscosity of plant extracts and increases the yield of yerba mate tea. This enzymatic action has a cascading effect, modifying multiple components, potentially releasing antioxidant compounds beyond phenolic compounds, and thereby influencing antioxidant activity. The combined action of Viscozyme and Pectinex significantly enhances the processing yield of caffeine, theobromine, caffeoylquinic acids, and the radical scavenging activities of yerba mate extracts. Importantly, the resulting enzymatic extracts from yerba mate tea are water-soluble and safe for consumption. For example, a 2-hour enzyme treatment substantially increased the content of caffeine, theobromine, caffeoylquinic acids, and radical scavenging activity. Accordingly, some embodiments of the present disclosure use a multi-enzyme complex including Viscozyme and Pectinex.

In some embodiments, the plant from which the plant extract is obtained is yerba mate. In some embodiments, the plant is one or more components of the cocoa pod, such as cocoa pod husk, cocoa bean, or cocoa pulp.

The type of protein powder in the composition may be any type of comestible, food-grade protein. In some embodiments, the protein powder is beef protein. In some embodiments, the protein powder is whey protein, soy protein, pea protein, or rice protein. In other embodiments, the protein powder is a combination of protein from one or more sources, such as from one or more animal sources and/or one or more plant sources.

In general, the plant extract may include any bioactive compound. In some embodiments, the plant extract includes polyphenols. Non-limiting examples of polyphenol flavonoid compounds include flavone, kaempferol, fisetin, apigenin, rutin, quercetin, naringin, naringenin, esculin, esculetin, biochanin A, and catechin. Non-limiting examples of polyphenol phenolic acid compounds include quercitinic acid, quinic acid, gallic acid, salicylic acid, coumaric acid, chlorogenic acid, caffeic acid, syringic acid, protocatechuic acid, sinapic acid, and 4-hydroxybenzoic acid.

In some embodiments, the plant extract includes alkaloid compounds. Non-limiting examples of alkaloids include methylxanthines and methylurates, such as caffeine, theobromine, theacrine, and theophylline.

In some embodiments, the plant extract includes triterpenoid saponin. Non-limiting examples of saponin include metasaponin 1, 2, 3, 4 and 5, with ursolic acid moieties.

In some embodiments, the composition further includes a resistance starch. The amount of resistance starch may vary widely according to the specific use. In some embodiments, the resistance starch is provided along with hydrolyzed protein. In some embodiments, a weight ratio of resistance starch to hydrolyzed protein is between about 1:2 to about 2:1. In some embodiments, the ratio of resistance starch to hydrolyzed protein is about 1:1.

In some embodiments, the composition includes a resistance maltodextrin. The amount of resistance maltodextrin in the composition may vary according to the particular use. For example, a weight ratio of the resistance maltodextrin relative to the plant extract may be between about 1:1 to about 1:5. In some embodiments, the resistance maltodextrin is in a weight ratio of 1 part resistance maltodextrin to 3 parts plant extract.

Some embodiments of the present disclosure include compositions for improving health and well-being in a subject. In some embodiments, the compositions include caffeine, theobromine, and/or total chlorogenic acids. In some embodiments, the compositions include the enzyme that was used during the process of extracting the plant extract. In some embodiments, the enzyme is deactivated. In some embodiments, the enzyme is present in an amount of between about 0.01% to about 5% w/w, such as 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, or 5% w/w, or an amount within a range defined by any two of the aforementioned values. In some embodiments, the caffeine is present in an amount ranging between 1.5% to about 5% w/w, such as an amount of 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0% w/w, or an amount within a range defined by any two of the aforementioned values, or less. In some embodiments, the theobromine is present in an amount of in an amount between 0.5% and about 1% w/w, such as 0.5, 0.6, 0.7, 0.8, 0.9, or 1.0% w/w, or an amount within a range defined by any two of the aforementioned values, or less. In some embodiments, the total caffeoylquinic acids are present in an amount ranging between about 5% and about 50% w/w, such as 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.1, 20.2, 20.3, 20.4, 20.5, 20.6, 20.7, 20.8, 20.9, 30, 30.1, 30.2, 30.3, 30.4, 30.5, 30.6, 30.7, 30.8, 30.9, 40, 40.1, 40.2, 40.3, 40.4, 40.5, 40.6, 40.7, 40.8, 40.9, 41, 41.1, 41.2, 41.3, 41.4, 41.5, 41.6, 41.7, 41.8, 41.9, 42, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44, 44.1, 44.2, 44.3, 44.4, 44.5, 44.6, 44.7, 44.8, 44.9, 45, 45.1, 45.2, 45.3, 45.4, 45.5, 45.6, 45.7, 45.8, 45.9, 46, 46.1, 46.2, 46.3, 46.4, 46.5, 46.6, 46.7, 46.8, 46.9, 47, 47.1, 47.2, 47.3, 47.4, 47.5, 47.6, 47.7, 47.8, 47.9, 48, 48.1, 48.2, 48.3, 48.4, 48.5, 48.6, 48.7, 48.8, 48.9, 49, 49.1, 49.2, 49.3, 49.4, 49.5, 49.6, 49.7, 49.8, 49.9, or 50% w/w, or an amount within a range defined by any two of the aforementioned values. In some embodiments, the composition is formulated as a powder or concentrated fluid.

In some embodiments, the enzyme is cellulase, xylanase, hemicellulase, or a combination thereof.

In some embodiments, the caffeine is present in an amount of less than about 50 mg per unit dose, such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg per unit dose, or an amount within a range defined by any two of the aforementioned values. The unit dose may vary according to the particular application; in some embodiments, the unit dose is between about 4 oz and about 20 oz, such as about 4, 5, 6, 7, 8, 9, 10, 11, 12, 13, 14, 15, 16, 17, 18, 19, or 20 oz, or an amount within a range defined by any two of the aforementioned values. In some embodiments, the unit dose is about 8 oz.

### Methods of Extracting a Plant Extract

Some embodiments provided herein relate to methods for extracting a plant extract from a plant material. Some embodiments provided herein relate to methods for producing a hydrolyzed protein. In some embodiments, the methods include providing a buffer solution at a pH of interest, mixing the plant material or the protein with the buffer solution to form a homogenized slurry, adding an enzyme to the homogenized slurry to form a slurry-enzyme mixture, heating the slurry-enzyme mixture to a first temperature, maintaining the slurry-enzyme mixture at the first temperature for a duration of time, heating the slurry-enzyme mixture to a second temperature, maintaining the slurry-enzyme mixture at the second temperature for a duration of time, and separating the slurry-enzyme mixture into a solid fraction and a liquid fraction. In some embodiments the liquid fraction includes the plant extract or the hydrolyzed protein. In some embodiments, the plant material or the protein and the buffer solution are present in a ratio between about 1:2 to about 1:20, such as about 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20, or at a ratio within a range defined by any two of the aforementioned values. In some embodiments, the first temperature is at an amount of between about 40° C. to about 60° C., such as about 40, 45, 50, 55, or 60° C., or an amount within a range defined by any two of the aforementioned values. In some embodiments, the slurry-enzyme mixture is maintained at the first temperature for a time period between about 1 hour and about 12 hours, such as 1, 2, 3, 4, 5, 6, 7, 8, 9, 10, 11, or 12 hours, or an amount of time within a range defined by any two of the aforementioned values. In some embodiments, the second temperature is a temperature of about 90° C. to about 99° C., such as 90, 91, 92, 93, 94, 95, 96, 97, 98, or 99° C., or a temperature within a range defined by any two of the aforementioned values. In some embodiments, the second temperature is maintained for a time period of between about 5 minutes to about 15 minutes, such as 5, 10, 15, or 60 minutes, or an amount of time within a range defined by any two of the aforementioned values.

In some embodiments, the plant material is any plant material that includes plant extracts that may be extracted. In some embodiments, the plant material includes yerba mate leaves. In some embodiments, the plant material includes cocoa pod husks.

In some embodiments, the protein used to produce a hydrolyzed protein is any protein of nutritional or health value. Some non-limiting examples of suitable proteins include beef protein, whey protein, soy protein, pea protein, and rice protein. In some embodiments, a combination of proteins suitable to be hydrolyzed may be used.

In some embodiments, the plant extract is any compound present in the plant material being extracted. For example, in some embodiments the plant extract is caffeine. Other, non-limiting examples of plant extracts used in some embodiments of the present disclosure include theobromine, rutin, chlorogenic acid, isomers of chlorogenic acid including 3-CQA, 4-CQA, 5-CQA, 3,4-diCQA, 3,5-diCQA, caffeoylquinic acids, polyphenols, or a combination thereof.

In some embodiments, the buffer solution is any appropriate buffer for maintaining the pH of interest. In some embodiments, the buffer solution includes citric acid, tricalcium citrate, and sodium hydroxide. In some embodiments, the buffer solution includes potassium dehydrate phosphate. In some embodiments, the buffer solution includes sodium acetate and acetic acid. In some embodiments, the buffer solution includes citric acid and sodium hydroxide. In some embodiments, the buffer solution includes potassium dihydrogen phosphate and potassium hydroxide. In some embodiments, the buffer solution includes potassium dihydrogen and sodium hydroxide. In some embodiments, the buffer solution includes disodium hydrogen phosphate and sodium dihydrogen phosphate. In some embodiments, the buffer solution includes dipotassium hydrogen phosphate and potassium dihydrogen phosphate. In some embodiments, the buffer solution includes potassium dihydrogen phosphate and disodium hydrogen phosphate. In some embodiments, the buffer solution includes dipotassium hydrogen phosphate and sodium dihydrogen phosphate. In some embodiments, the buffer solution includes anhydrous disodium hydrogen phosphate and citric acid. In some embodiments, the buffer solution includes anhydrous dipotassium hydrogen phosphate and citric acid.

In some embodiments, the pH of interest is any appropriate pH for extraction of the plant material or for the hydrolysis of a protein. The pH may vary according to the specific use; for example, it may depend on the specific activity of the enzyme or enzymes used for extraction of the plant material, as the extraction activity as a function of pH may vary from enzyme to enzyme. In some embodiments, the pH of the buffer solution is about 5.0. In some embodiments, the pH of the buffer solution is about 5.5. In some embodiments, the pH of the buffer solution is about 5.8. In some embodiments, the pH of the buffer solution is about 7.0. In some embodiments, the pH of the buffer solution is about 8.0.

The mixing of the plant material or a protein with the buffer solution to form a homogenized slurry may be achieved by any means known to the skilled person. Generally, the mixing involves subjecting the plant material or protein to physical mixing, such as in a batch process having a stainless steel tank reactor and stirrer. In some embodiments, more sophisticated physical mixing techniques, such as sonication, may be used.

Some embodiments of the disclosed method include adding an enzyme to the homogenized slurry to form a slurry-enzyme mixture. The enzyme may be any enzyme that is appropriate for extracting plant extracts from a plant material, or for hydrolyzing a protein. Some non-limiting examples of enzymes used include a cellulase, hemicellulase, pectinase, α-amylase, β-amylase, xylanase, β-glucanase, protease, phytase, esterase, endo-β-1,4-glucanase, cellobiohydrolase, proteinase, or a combination thereof.

In some embodiments, the enzyme is added in any appropriate amount such as to increase the activity of the enzyme toward extracting the plant material or to increase the activity of the enzyme toward hydrolyzing the protein. In some embodiments, the enzyme is added in an amount between about 0.01% w/w to about 5% w/w, such as in an amount of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, or 5% w/w, or an amount within a range defined by any two of the aforementioned values.

In some embodiments, more than one enzyme is added to the homogenized slurry in order to form the slurry-enzyme mixture. In some embodiments, two or more enzymes are added, such as to form, for example, an enzyme cocktail. In some embodiments, the use of more than one enzyme may have many advantages, such as, for example, acting on different components of the plant cell wall. In some embodiments, the more than one enzymes may be added simultaneously to the homogenized slurry, or they may be added sequentially, such as after a period of minutes or hours after the first enzyme is added. Without being bound by theory, it is believed that by adding different enzymes sequentially to the homogenized slurry, the different enzymes may have a synergistic effect on degrading the plant cell wall and thus increasing extraction efficiency.

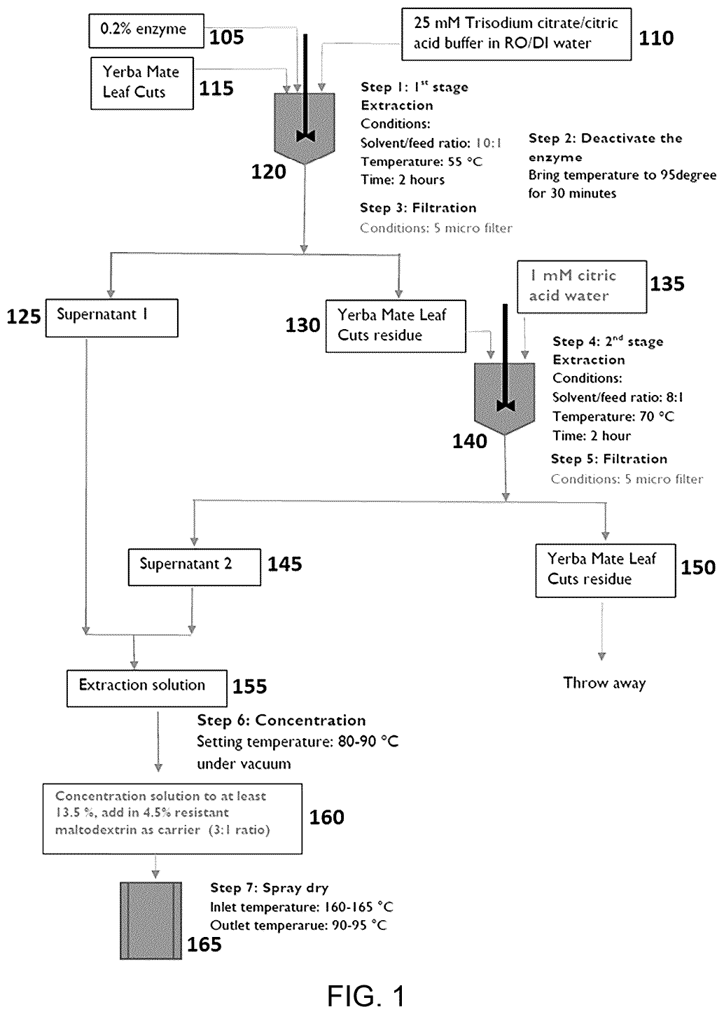

An example flow-chart representation of the methods as contemplated by some embodiments of the present disclosure is provided in FIG. 1. 0.2% of an enzyme 105, 25 mM of trisodium citrate/citric acid buffer in RO/DI (reverse osmosis/de-ionized) water 110, and yerba mate leaf cuts 115 are added to a steel tank reactor with stirrer 120. The first stage extraction occurs in the tank reactor 120, under conditions where the solvent/feed ratio is 10:1, the temperature is 55° C., and the time of reaction is 2 hours. Following the first stage extraction, the product is separated into a solid phase, yerba mate leaf cuts residue 130, and the liquid phase supernatant 1 125. The separation is achieved by a filtration through a 5 micro filter.

The yerba mate leaf cuts residue 130 and 1 mM citric acid in water 135 are added to a second tank reactor 140, where the second tank reactor 140 may be the same or different as the reactor 120. The second stage extraction occurs in the tank reactor 140, under conditions where the solvent/feed ratio is 8:1, the temperature is 70° C., and the time of reaction is 2 hours. Following the second stage extraction, the product is again separated into a second supernatant 145 and yerba mate leaf cut residue 150, as achieved by filtration through a 5 micro filter.

The yerba mate leaf cuts residue 150 is discarded. The supernatant 2 145 and supernatant 1 125 are combined to form an extraction solution 155. The extraction solution 155 is concentrated under vacuum at a temperature of 80° C. to 90° C., to form a solution concentrated to at least 13.5%, to which 4.5% resistant maltodextrin as carrier is added 160. Spray drying is performed on concentrated solution 160 in spray dryer 165, with an inlet temperature of 160-165° C. and outlet temperature of 90-95° C.

In some embodiments, the method for making hydrolyzed proteins includes providing a protein solution that includes protein, adjusting a pH of the protein solution to basic to form a basic protein solution, adding a protease enzyme to the basic protein solution, and incubating the basic proteins solution and the protease enzyme for a time period sufficient to convert at least a portion of the protein in the solution to hydrolyzed protein.

In some embodiments, the methods for extracting a plant extract described herein result in increased concentrations of extracted bioactive molecules, such as polyphenols. In some embodiments, the extracted products using the methods described herein are formulated into the compositions described herein, complete with the enzymes used in the extraction process. In some embodiments, the formulations are administered or provided to a subject, or ingested by a subject. The formulations provided herein provide beneficial health outcomes, including reduced inflammation, improving heart health, and improving well-being in a subject.

### Microencapsulation of a Plant Extract

Spray-drying is a process used in various industries, including food and pharmaceuticals, where a liquid product with solids is turned into a powdered form. This may be accomplished by spraying the liquid into a heated stream of drying gas, often using air, but sometimes using inert gases like nitrogen. The main advantages of spray-drying are its cost-effectiveness, scalability, continuous operation capability, and minimal risk of thermal damage to the product. Additionally, it can create microencapsulated products, where tiny particles are enclosed in a protective coating or embedded in a uniform or non-uniform matrix (Ameri and Maa, 2006; Patel, Patel, and Suthar, 2009; Wang and Langrish, 2009; Cal and Sollohub, 2010; Murugesan and Orsat, 2012; Shishir and Chen, 2017; Santos et al., 2018).

Microencapsulation, achieved through techniques like spray-drying, has opened up new possibilities in product development (I Ré, 1998; Gharsallaoui et al., 2007; Samborska et al., 2021). The protective coating, also known as the wall, coating, or carrier material, helps safeguard the enclosed core material. The core material can include one or more ingredients, in the form of a solution, emulsion, or suspended solids or smaller microcapsules. The coating material itself can also be a combination of different materials.

Microencapsulation finds applications in the food and pharmaceutical sectors for several purposes. Microencapsulation can slow down the release of the core material into the environment, making it useful for controlled release applications. Microencapsulation makes handling easier and allows for precise dilution of the core material to achieve the desired concentration. Another important role of microencapsulation, especially in spray-drying, is moisture removal and reducing water activity. This contributes to the stability of the product by preventing microbial and biochemical reactions, cutting down on storage and transportation costs, and enhancing specific quality attributes like flowability, solubility, and hygroscopicity (Amaral, Andrade and Conto, 2019; Yang et al., 2020; Li et al., 2023).

Carbohydrates like maltodextrin, inulin, and lactose have been widely used as encapsulating agents due to their generally low viscosity at high concentrations and good solubility in water, but they often lack the good interfacial properties associated with gums and proteins. Maltodextrin is a polymer of D-glucose obtained from acid or enzymatic hydrolysis of corn starch, and is available in different dextrose equivalents (DE), which correspond with the degree of hydrolysis of the starch. The DE is therefore also inversely related to the average molecular weight. Maltodextrin is commonly used in spray drying due to its neutral smell and taste, high solubility in cold water, low cost, low hygroscopicity, and low viscosity at high concentrations. It has also been shown to exhibit an antioxidant effect and good volatile retention. It lacks good emulsifying properties, but this can be addressed by the addition of a second carrier which does provide that function, like gum arabic (Costa et al., 2015). Gum arabic, a polysaccharide containing mainly D-glucoronic acid, D-galactose, L-rhamnose, and L-arabinose, is one of the most commonly used carriers due to its excellent solubility, low viscosity, high oxidative stability, and good emulsifying properties, which is attributed to a small protein fraction (2%) found among its constituents. This makes it an excellent choice for the encapsulation of lipids. High costs and limited supplies often prohibit the more widespread use of gum arabic, however, and alternatives have been proposed, e.g. mesquite gum.

Resistant maltodextrin (RMD) is a unique form of dietary fiber categorized as resistant starch type V. It is produced by modifying starch through debranching processes. Structurally, it consists of alpha-glucoside oligosaccharides with an average degree of polymerization ranging from 10 to 15. Resistant maltodextrin is a type of fiber that is not fully digested by the body. This means that it passes through the small intestine without being absorbed into the bloodstream, and instead travels to the colon, where it is fermented by bacteria. One of the key benefits of RMD is that it does not cause spikes in blood sugar levels. This is because RMD is not broken down into monosaccharides, which are the simplest form of sugar and are easily absorbed into the bloodstream. When monosaccharides are absorbed into the bloodstream, they cause blood sugar levels to rise rapidly. Instead, RMD is fermented by bacteria in the colon, which produces short-chain fatty acids (SCFAs). SCFAs are beneficial for gut health and have been shown to improve blood sugar control. This property makes it a valuable choice as a carrier material for antidiabetic nutraceuticals. Using a carrier with a high glycemic index in such formulations would counteract the hypoglycemic effects of bioactive compounds. One of the key benefits of RMD is that it acts as a prebiotic. This means that it promotes the growth of beneficial bifidobacteria in the gut. Bifidobacteria are a type of bacteria that are important for gut health. They help to keep the gut lining healthy and fight off harmful bacteria. Increased consumption of dietary fiber, particularly prebiotic fibers like RMD, has been associated with a number of health benefits, including: reduced risk of cardiovascular diseases, lower body mass index, improved gut health, reduced risk of type 2 diabetes, improved immune function and reduced risk of colon cancer. RMD is a versatile ingredient suitable for incorporation into various foods and beverages, such as yogurt, smoothies, baked goods, and cereal. It is also available as a standalone supplement.

One of the key advantages of RMD in spray drying applications is its suitability as a wall material. Several factors make it ideal for this purpose. RMD readily dissolves in water, allowing it to create a continuous protective layer around the core. Its low viscosity simplifies the atomization process during spray drying, making it an efficient choice for this technique. RMD is relatively affordable, which is crucial for commercial applications. Its bland taste and odor make it versatile for use in a wide range of food and beverage products.

Fibersol-2 is a digestion-resistant maltodextrin that is produced and marketed through a partnership between ADM and Matsutani LLC. This ingredient has received recognition as Generally Recognized as Safe (GRAS). It has also been approved by the Japanese Ministry of Health, Labour, and Welfare as a “Standardized FOSHU (Food for Specified Health Uses)” ingredient. Specifically, it contains the bioactive compound known as digestion-resistant maltodextrin. In the context of FOSHU, Fibersol-2 serves designated functions, including acting as a dietary fiber to promote regularity in intestinal function and to help control post-meal blood glucose and triglyceride levels. The FOSHU dosage recommendations are 3-8 g of Fibersol-2 as dietary fiber for improving intestinal regularity and 4-6 g for controlling blood glucose levels (Martirosyan and Chen, 2021).

Fibersol-2, also known as “resistant maltodextrin” (RMD), “nondigestible dextrin,” or “indigestible dextrin,” is created through a controlled enzymatic process involving corn starch molecules. This process replaces the typical alpha-1,4-linkages in corn starch with alpha and beta 1,2-, 1,3-, 1,4-, and 1,6-linkages, rendering it resistant to digestion (Ohkuma and Wakabayashi, 2008). It is available in the form of a tasteless, water-soluble powder or liquid that can be seamlessly incorporated into food and beverages without altering their taste or texture. Notably, in Japan, there are over 275 products that have received FOSHU approval because they contain Fibersol-2. This recognition underscores its potential significance in addressing health concerns related to the modern Western diet, which tends to be deficient in fiber. Given the rising incidence of nutrition-related diseases such as diabetes and obesity, Fibersol-2 holds promise as a valuable tool for both prevention and symptom management.

In some embodiments, any of the compositions and formulations described herein include Fibersol. In some embodiments, the compositions and formulations result in enhanced energy by sustaining energy and vitality. In some embodiments, the compositions and formulations improve gut health by promoting healthy gut microbiome by nourishing beneficial bacteria.

### Numbered Embodiments

Further provided are exemplary embodiments of the methods and compositions described herein.

1. A composition, comprising:
an enzyme;
a plant extract obtained from a plant or a hydrolyzed protein powder,
wherein the composition is formulated as a powder or a concentrated fluid.

2. The composition of embodiment 1, wherein the enzyme is present in an amount of about 0.01% to about 5% w/w, and wherein the enzyme is configured as a processing aid.

3. The composition of any one of embodiments 1-2, wherein the enzyme is deactivated.

4. The composition of any one of embodiments 1-3, wherein the enzyme is a cellulase, hemicellulase, pectinase, α-amylase, β-amylase, xylanase, β-glucanase, protease, phytase, esterase, endo-β-1,4-glucanase, cellobiohydrolase, proteinase, or a combination thereof.

5. The composition of any one of embodiments 1-4, further comprising one or more endo-β-glucanases or one or more polygalacturonases.

6. The composition of any one of embodiments 1-5, wherein the plant is yerba mate or cocoa.

7. The composition of any one of embodiments 1-6, wherein the hydrolyzed protein powder is beef protein, whey protein, soy protein, pea protein, or rice protein.

8. The composition of any one of embodiments 1-7, wherein the plant extract is caffeine, theobromine, caffeoylquinic acid, rutin, triterpenoid, or chlorogenic acid, or a combination thereof.

9. The composition of any one of embodiments 1-8, wherein the rutin is present in an amount of about 0.5% to about 2%.

10. The composition of any one of embodiments 1-9, further comprising resistance maltodextrin.

11. The composition of embodiment 10, wherein the resistance maltodextrin is present in a ratio of 1 part resistance maltodextrin to 3 parts plant extract.

12. The composition of any one of embodiments 1-11, further comprising resistance starch.

13. The composition of embodiment 12, wherein the resistance starch is present in a ratio of 1 part resistance starch to 1 part hydrolyzed protein.

14. The composition of any one of embodiments 1-13, wherein the plant extract is spray-dried with a solution comprising resistant maltodextrin.

15. The composition of any one of embodiments 1-14, wherein the plant extract is spray-dried with a solution comprising resistant starch.

16. The composition of any one of embodiments 1-15, wherein the plant extract is spray-dried with a solution comprising resistant dextrin.

17. A composition for improving health and well-being in a subject, comprising:
an enzyme present in an amount of about 0.01% to about 5% w/w, wherein the enzyme is cellulase, xylanase, or hemicellulase;
caffeine present in an amount of between 1.5% and 5% w/w;
theobromine present in an amount of at least 0.5% w/w; and
total caffeoylquinic acids present in an amount ranging from about 5% to about 50% w/w,
wherein the composition is formulated as a powder or a concentrated fluid.

18. The composition of embodiment 17, further comprising resistant maltodextrin (RMD).

19. The composition of embodiment 18, wherein the RMD is spray-dried on the powder or the concentrated fluid.

20. The composition of any one of embodiments 17-19, wherein the caffeine is present in an amount less than about 50 mg per unit dose.

21. A method for extracting a plant extract from a plant material or for producing a hydrolyzed protein, the method comprising:
providing a buffer solution;
   mixing the plant material or a protein with the buffer solution to form a homogenized slurry;
adding an enzyme to the homogenized slurry to form a slurry-enzyme mixture;
heating the slurry-enzyme mixture to a first temperature;
maintaining the slurry-enzyme mixture at the first temperature;
heating the slurry-enzyme mixture to a second temperature;
maintaining the slurry-enzyme mixture at the second temperature; and
separating the slurry-enzyme mixture into a solid fraction and a liquid fraction, wherein the liquid fraction comprises the plant extract or the hydrolyzed protein.

22. The method of embodiment 21, wherein the buffer solution comprises citric acid, tricalcium citrate, or sodium hydroxide.

23. The method of embodiment 21, wherein the buffer solution comprises potassium dihydrogen phosphate or dipotassium hydrogen phosphate.

24. The method of any one of embodiments 21-23, wherein the pH of the buffer solution is about 5.8.

25. The method of any one of embodiments 21-23, wherein the pH of the buffer solution is between about 5.0 and about 6.2.

26. The method of any one of embodiments 21-23, wherein the pH of the buffer solution is greater than 7.

27. The method of any one of embodiments 21-26, wherein the plant material or protein is in a ratio with the buffer solution in an amount of between 1:2 and 1:20.

28. The method of any one of embodiments 21-27, wherein the first temperature is in a range from about 40° C. to about 60° C.

29. The method of any one of embodiments 21-28 wherein the first temperature is maintained for a period of time ranging from about 1 hour to about 12 hours.

30. The method of any one of embodiments 21-29, wherein the second temperature is in a range from about 90° C. to about 99° C.

31. The method of any one of embodiments 21-30, wherein the second temperature is maintained for a period of time ranging from about 5 minutes to about 15 minutes.

32. The method of any one of embodiment 21-31, wherein the plant material comprises yerba mate leaves.

33. The method of any one of embodiments 21-32, wherein the plant material comprises cocoa pod husk.

34. The method of any one of embodiments 21-33, wherein the protein comprises beef protein.

35. The method of any one of embodiments 21-34, wherein the plant extract is caffeine, theobromine, rutin, chlorogenic acid, an isomer of caffeoylquinic acids (CQA), polyphenols, saponin, or a combination thereof.

36. The method of embodiment 35, wherein the isomer of CQA comprises 3-CQA, 4-CQA, 5-CQA, 3,4-diCQA, 3,5-diCQA, or 4,5-diCQA.

37. The method of any one of embodiments 21-36, further comprising adding a second enzyme to the homogenized slurry.

38. The method of embodiment 37, wherein the enzyme is Viscozyme and the second enzyme is Pectinex.

39. The method of embodiment 37, wherein the enzyme is Alcalase and the second enzyme is Flavourzyme.

40. The method of any one of embodiments 37-39, wherein the enzyme and the second enzyme are added simultaneously or sequentially.

41. The method of any one of embodiments 21-40, wherein the enzyme is a cellulase, hemicellulase, pectinase, α-amylase, β-amylase, xylanase, β-glucanase, protease, phytase, esterase, endo-β-1,4-glucanase, cellobiohydrolase, proteinase, or a combination thereof.

42. The method of any one of embodiments 21-41, wherein the enzyme is added in an amount ranging from about 0.01% to about 5% w/w.

43. The method of any one of embodiments 21-42, further comprising spray-drying the plant extract with a solution comprising resistant maltodextrin (RMD).

44. A method for improving health or well-being in a subject, comprising:
ingesting a composition comprising a plant extract obtained from a plant or a hydrolyzed protein powder.

45. The method of embodiment 44, wherein improving health or well-being comprises improving blood lipid levels, reducing body weight, reducing fasting blood sugar levels, increasing feelings of positive experiences, or decreasing feelings of negative experiences.

46. The method of any one of embodiments 44-45, wherein the composition comprises:
caffeine present in an amount of between 1.5% and 5% w/w;
theobromine present in an amount of at least 0.5% w/w; and
total caffeoylquinic acids present in an amount ranging from about 5% to about 50% w/w.

47. The method of any one of embodiments 44-46, wherein the composition is formulated as a powder or a concentrated fluid.

48. The method of any one of embodiments 44-47, wherein the plant is yerba mate or cocoa.

49. The method of any one of embodiments 44-48, wherein the hydrolyzed protein powder is beef protein, whey protein, soy protein, pea protein, or rice protein.

50. The method of any one of embodiments 44-49, wherein the plant extract is caffeine, theobromine, caffeoylquinic acid, rutin, or chlorogenic acid, saponin, or a combination thereof.

51. The method of any one of embodiments 44-50, wherein the composition further comprises resistance maltodextrin.

52. The method of embodiment 51, wherein the resistance maltodextrin is present in a ratio of 1 part resistance maltodextrin to 3 parts plant extract.

53. The method of any one of embodiments 44-52, wherein the composition further comprises resistance starch.

54. The method of embodiment 53, wherein the resistance starch is present in a ratio of 1 part resistance starch to 1 part hydrolyzed protein.

55. The method of any one of embodiments 44-54, wherein the plant extract is spray-dried with a solution comprising resistant maltodextrin.

56. The method of any one of embodiments 44-55, wherein the plant extract is spray-dried with a solution comprising resistant starch.

57. The method of any one of embodiments 44-56, wherein the plant extract is spray-dried with a solution comprising resistant dextrin.

58. A method of extracting a plant extract from plant material comprising:
providing plant material;
treating said plant material with one or more enzymes comprising a pectinase, a hemicellulase, a beta-glucanase, or a xylanase; and
extracting the plant extract from the treated plant material, wherein the extraction is conducted at a pH of 5-6.

59. The method of embodiment 58, wherein the plant material is yerba mate.

60. The method of embodiment 58 or embodiment 59, wherein the plant extract is a saponin.

61. The method of embodiment 61, wherein the saponin is matesaponin 1, matesaponin 2, matesaponin 3, matesaponin 4, matesaponin 5, mate saponin A, mate saponin B, ursolic acid and derivatives thereof, or oleanolic acid or derivatives thereof.

62. The method of any one of embodiments 58-61, wherein the one or more enzymes comprise a first blend comprising pectinase, hemicellulase, and beta-glucanase and a second blend comprising beta-glucanase, pectinase, hemicellulase, and xylanase.

63. The method of any one of embodiment 62, wherein the first blend comprises Pectinex® and the second blend comprises Viscozyme®.

64. The method of any one of embodiments 58-63, further comprising breaking down yerba mate cell walls and releasing saponins.

65. The method of any one of embodiments 62-64, wherein the first blend and second blend are used in a ratio of about 1:1.

66. The method of any one of embodiments 58-65, wherein the one or more enzymes are used at a concentration of about 0.5%.

67. The method of any one of embodiments 58-66, wherein the extraction yields a saponin content of at least 10%.

68. The method of any one of embodiments 58-67, wherein the extracted saponins exhibit antioxidant activity as measured by DPPH.

### EXAMPLES

Some aspects of the embodiments discussed above are disclosed in further detail in the following examples, which are not in any way intended to limit the scope of the present disclosure. Those in the art will appreciate that many other embodiments also fall within the scope of the disclosure, as it is described herein above and in the claims.

### Example 1: Yerba Mate Leaf Cut Enzymatic Extraction

The yerba mate tea leaves (2-5 mm cut) used in this example were sourced from Qualita (Brazil). Citric acid monohydrate and Sodium citrate were purchased from TCI chemicals. High performance liquid chromatography (HPLC) grade water, methanol, acetonitrile, LC (liquid chromatography) grade formic acid were purchased from Sigma (St. Louis, MO, USA). Reference standards chlorogenic acid, caffeine, theobromine, rutin, 3,4-dicaffeoylquinic acid, 3,5-dicaffeolyquinic acid, 4,5-dicaffeoylquinic acid were purchased from Sigma (St. Louis, MO, USA). DPPH (2,2-diphenyl-1-picrylhydrazyl), product of Caymon Chemicals, was purchased from i-DNA Biotechnology, SG.

Viscozyme L (from Aspergillus aculeatus, 100 fungal β-glucanase units (FBG)/ml, Novozymes, Bagsvaerd, Denmark), Celluclast 1.5 L (from Trichoderma reesei, 700 endoglucanase units (EGU)/ml, Novozymes, Bagsvaerd, Denmark), and Pectinex 5XL (from Aspergillus niger, 5000 ferment depectinization units (FDU)/ml, Novozymes, Bagsvaerd, Denmark) were used.

The extraction process used in this example is summarized in FIG. 1. The enzymatic extraction involved: 1. Treating yerba mate leaves with an aqueous enzyme solution to degrade the yerba mate plant tissue; 2. Extraction of the enzyme treated materials by water; 3. Separation of the solid residue from the aqueous solution that contained the bioactive compounds; and 4. Recovering the bioactive compounds by vacuum evaporation and spray drying.

In this example, various reaction variables were independently examined, including the enzyme-to-substrate ratio, which ranged from 0 to 2 (equivalent to 0 to 200 FBG for ViscozymeL, 0 to 70 EGU for Celluclast 1.5 L, and 0 to 500 FDU for Pectinex 5XL). Additionally, parameters such as reaction solution pH ranging from 3.0 to 6.0), reaction temperature (from 40° C. to 60° C.), and reaction time (from 1 to 20 hours) were investigated.

The process involved dispersing 100 grams of dried yerba mate leaves in either 1 liter of water or a buffer solution adjusted to the optimal pH using sodium citrate/citric acid buffer or potassium dihydrogen phosphate buffer. This suspension was thoroughly mixed using both an overhead mixer and a magnetic stirrer hotplate, with the temperature maintained at a constant level throughout. After homogenization, the pH was checked and, if necessary, adjusted to maintain stability at the optimal conditions, which, in this case, was a pH of 5.0-6.0. The enzyme preparation was then added to the suspension.

The hydrolysis and extraction process occurred at a temperature of 50° C. for a duration of 2 hours. To stop the enzymatic activity, the sample was subjected to boiling at 95° C. for 10 minutes, followed by rapid cooling. Finally, the resulting extract was filtered through a 5 μm filter cloth to eliminate any remaining solids or impurities.

### Example 2: Spray Drying and Analysis of Bioactive Compounds

Spray-drying was conducted using a Mini spray-dryer at the laboratory scale (OLD-SD8000B, Xiamen Ollital). To facilitate the drying process, pressurized air served as the drying medium. The spray drying parameters used were determined as follows: air inlet temperature 140-150° C., air outlet temperature 92.3° C. The extracts were delivered into the spray dryer by using a peristaltic pump at a rate of 15-16 g/min. The feed rate was controlled to maintain air outlet temperature stabilized at desired level. The spray dried powders were collected in a pre-warmed glass collection bottle attached at the bottom of the cyclone. To avoid collection of the transitional sample between batches, the powder discharged at the first and last 5 min of each batch was not collected. Powder adhered to the spray dryer chamber inner wall and other parts of the spray dryer components were not collected. The powder samples were sealed in airtight packaging while awaiting further analysis. At the conclusion of each experimental run, once the entire volume of the prepared extract feed was transformed into powder, the heating of the inlet air was discontinued. The system continued to operate until the inlet temperature dropped below 80° C. At that point, the system was turned off, and the extracts were recovered from the collection vessel. Throughout the spray-drying process, the average outlet temperature was continuously monitored. This temperature was measured using an in-line temperature probe situated between the outlet of the spray-cylinder and the cyclone system. The outlet air temperature for each experimental run was recorded after the spraying process had been running for a minimum of 5 minutes to ensure that it had reached a stable state. The reported temperature value represented the mean of three measurements taken at 5-minute intervals.

Quantitative analysis of bioactive compounds was carried out using a Thermo Fisher Vanquish series HPLC system, which included chromeleon software, a VC-P10-A-01 pump, and a VC-D11-A-01 diode array detector (DAD). The separation of compounds was performed through liquid chromatography on a reversed-phase Accucore C18 column (150 mm×2.1 mm, particle size 2.6 μm, Thermo Fisher) at a constant temperature of 30° C. Caffeine and theobromine were detected at a wavelength of 280 nm, while rutins and other chlorogenic acids were detected at 350 nm. The flow rate was set at 0.4 ml/min, and each injection contained 10 μl of the sample.

The mobile phase consisted of two components: 0.1% formic acid in water (A) and 0.1% formic acid in acetonitrile (B). The gradient elution of the mobile phase followed this pattern: 5-20% B in 0-10 min, 20% B in 10-12 min, 20-5% B in 12-15 min, 5-95% B in 15-15.1 min, 95% B in 15.1-16.5 min, 95-5% B in 16.5-16.6 min, and 5% B in 16.6-19 min. Prior to analysis, both the tested samples and standards were dissolved in HPLC grade water and filtered through 0.22 μm syringe filter. The identification of bioactives in yerba mate was completed by comparing the retention time with reference standards. The peak area of each component in the extracts was acquired from its chromatogram and the abundance of each compound was calculated from its corresponding calibration curve. Experiments were conducted in triplicate.

The method underwent a comprehensive validation process, including assessments for linearity, accuracy, precision, limits of detection (LOD), and quantification (LOQ). Mixed standards containing caffeine, 5-chlorogenic acid, chlorogenic acid, rutin, and 3,5-dicaffeoylquinic acid in water were prepared within a concentration range of 2-600 μg/ml. For theobromine, a separate solution was made by dissolving it in 1% NaOH and calibrated within the range of 3-500 μg/ml. Eight different concentrations of these solutions were injected in triplicate. Linear regression equations were established by plotting the peak area (y) against the concentration (x, mg/mL) to determine linearity. To establish the limits of detection (LOD) and quantification (LOQ), the mixed standards solution was further diluted to specific concentrations. LOD and LOQ were determined based on a signal-to-noise ratio of 3 and 10, respectively. Precision was evaluated both intra-day and inter-day. Intra-day precision involved continuously injecting the sample solution in triplicate on the same day. Inter-day precision was determined by measuring the solution once a day for three consecutive days. To assess accuracy, a recovery test was conducted using the standard addition approach. This involved adding known amounts of the analytes to the sample and then measuring the recovery percentage.

Repeatability was estimated by calculating the relative standard deviation (RSD) based on repeated measurements. The results are listed in Table 1. The 6 compounds showed excellent linearity with R²>0.998. A high recovery (91.2-96.8%) and precision were acceptable with RSD values ranging between 1.9% and 2.9% for intra-day variation. This validated method was successfully applied to the quality control of yerba mate extracts, which provided particularly important information for production and application.

TABLE 1
Peak assignment, linear regression equation, coefficients, linear range, precisions,
and recovery yields, limit of detection and limit of quantification of 6 standards

| | Retention | Regression | | Range | LOD | LOQ | Precision | Recovery |
|---|---|---|---|---|---|---|---|---|
| Identification | time (min) | equation | R² | (ug/mL) | (ug/mL) | (ug/mL) | (%) | (%) |
| Theobromine | 1.4 | y = 99095x − | 100.0 | 1.9-245 | 1.4 | 4.3 | 2.1 | 95.8 |
| | | 19.8 | | | | | | |
| Caffeine | 4.6 | y = 90975x + | 99.8 | 24-3157 | 1.0 | 3.1 | 1.9 | 96.8 |
| | | 137 | | | | | | |
| 5-caffeoyl- | 2.1 | y = 44745x − | 99.9 | 2-266 | 1.2 | 3.5 | 2.5 | 93.1 |
| quinic acid | | 17.3 | | | | | | |
| Chlorogenic | 4.2 | y = 50535x − | 99.9 | 4.8-613 | 1.0 | 3.1 | 1.9 | 95.2 |
| acid | | 106 | | | | | | |
| Rutin | 10.2 | y = 40255x + | 99.8 | 0.9-110 | 0.8 | 2.5 | 2.7 | 92.7 |
| | | 15.3 | | | | | | |
| 3,5-di-caffeoyl- | 11.8 | y = 64454x + | 99.9 | 0.36-466 | 1.0 | 3.1 | 1.9 | 91.2 |
| quinic acid | | 27.3 | | | | | | |

The scavenging activity of the extract again DHHP (2,2 diphenyl-1-picrylphydrazyl) radical was assessed according to reference method with some modification (Kedare and Singh, 2011). In brief, the plant extract's stock solution was initially prepared in methanol at concentration of 2000 μg/ml. Subsequently, a series of two-fold dilutions were carried out to generate various concentrations, including 1000, 500, 250, 125, 62.5, 31.25, and 15.62 μg/ml. For each diluted extract solution (2 ml), it was combined with 2 ml of a DPPH methanol solution at a concentration of 80 μg/ml. This mixture was then left in darkness at room temperature for 30 minutes. After the incubation period, the absorbance was measured using a spectrophotometer set at a wavelength of 517 nm. Control samples were also prepared, consisting of 2 ml of methanol mixed with 2 ml of the DPPH solution. In addition to this, chlorogenic acid was utilized as positive controls. The scavenging activity of the extract was quantified as the inhibition percentage, which is calculated using the following equation:

$$Scavenging activity ( \% ) = 100 \times [ ( {A}_{contro1} - {A}_{sample} ) / {A}_{control} ) ]$$

where A_{control} is the absorbance of DPPH radical in methanol and Aₛₐₘₚₗₑ is the absorbance of DPPH radical solution mixed with sample extract. An IC50 (half-maximal inhibitory concentration) value indicating the concentration at which a sample would inhibit free radicals by 50% was also calculated. All determinations were performed in triplicate.

The total phenolic content (TPC) of the extract was determined using Folin-Ciocalteu reagent (Ainsworth and Gillespie, 2007; Lamuela-Raventós, 2018). In brief, a solution of extract (0.5 ml) with proper dilution was mixed with 1.0 ml Folin Ciocalteu reagent at room temperature for 5 min and added 2 ml of 7% Na₂CO₃ solution. The mixture was boiled for 1 min, and absorbance of the color was recorded at 750 nm in spectrophotometer. The results are expressed in g gallic acid equivalent (GAE/g), on dry weight basis.

Representative sample of weight 1 gram (W0) from each batch was spread evenly in sample pan and placed into Moisture analyzer (Mettle Toledo HC103) to determine the moisture content. The heating temperature was set at 140° C. The heating stopped automatically as the dry weight of the sample was stable. The final weight of the sample (Wf) was then read. The moisture content was calculated as follows: Moisture content=(W0−Wf)/W0×100%.

The spray dryer configuration used in this example is capable of producing particles of typical mean size in the range of 5 to 25 μm. Sizing of particles at this size range can be examined by using a light microscope connected to an image analysis system. At least 250 particles to be measured for each batch and the mean particle diameter are computed. As the subtle difference among individual sample was not of a critical concern at current stage of study, the size analysis was not carried out at this stage until further study required.

The yield of the process was calculated as follows:

Yield=Weight of powder in collection bottle/Total solids in feed×100%.

Bulk density, compressibility index and Hausner ratios: Samples of 3 to 5 gram (M) from each batch were introduced gently and evenly into a 10-ml dry graduated cylinder without compacting. The unsettled apparent volume, V0, was read to the nearest graduated unit. It was then tapped manually until the difference between the two succeeding measurements was less than 2%. The final tapped volume, Vf, was then read. The bulk density (DB) and tapped density (DT) were calculated by formula: DB=M/V0, DT=M/Vf.

The Compressibility Index (CI) and Hausner ratio (HR) were calculated subsequently as follows:

$$CI = ( V 0 - Vf ) / V 0 \times 100 ;$$

$$HR = V 0 / Vf .$$

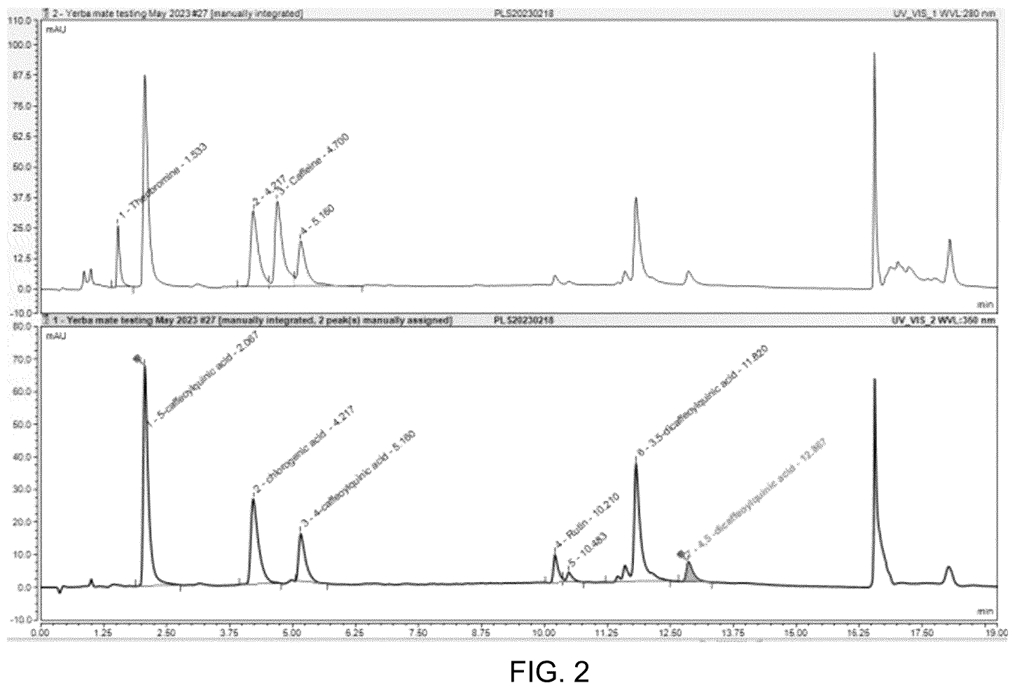

FIG. 2 displays a typical HPLC chromatogram of yerba mate extracts. In this chromatogram, theobromine and caffeine, two key compounds, exhibit their maximum absorption at 280 nm, and their identification and quantification were accomplished by employing reference standards. On the other hand, other phenolic acids and rutin have their maximum absorption at 350 nm. Among these compounds, 5-caffeoylquinic acid, chlorogenic acid, and 4-caffeoylquinic acid are the major caffeoylquinic acids found in yerba mate leaves. For 5-caffeoylquinic acid and chlorogenic acid, both identification and quantification were based on their respective reference standards. In the case of 4-caffeoylquinic acid, identification was achieved by considering its elution profile on C18 HPLC columns, as reported in the literature for CGA (chlorogenic acid) isomers found in plant foods, along with its UV spectra (Fang, Yu, and Prior, 2002).

In this example, 3,5-dicaffeoylquinic acid was identified and quantified using reference standards, while 4,5-dicaffeoylquinic acid was identified based on its elution profile as reported in the literature (Heck and Mejia, 2007). Quantification of 4,5-dicaffeoylquinic acid was achieved by using the calibration curve of 3,5-dicaffeoylquinic acid. To determine the total content of caffeoylquinic acids, the sum of chlorogenic acid isomers and dimers was calculated.

### Example 3: Effect of Enzymes on Extraction Efficiency

Plant bioactive compounds are often bound to cell wall polysaccharides through hydrophobic interactions and hydrogen bonds. Plant cell walls are structurally complex, consisting of three distinct layers, each composed of different polysaccharides. The first layer, known as the middle lamella, is primarily made up of pectin. The second layer, called the primary cell wall, contains a cellulose-xylogulan structure embedded in a matrix of pectin and structural proteins. The third layer, known as the secondary cell wall, is characterized by parallel cellulose microfibrils accompanied by hemicellulose, pectins, and lignin. To improve the extraction of bioactive compounds, it is essential to break the bonds within these plant cell polysaccharides and break down the structural polysaccharides in the various layers of the cell wall structure.

Cellulases, pectinases, glucanases, and their combinations are effective enzymes for hydrolyzing these bonds in plant cell polysaccharides. For cellulase to access cellulose, it is necessary to remove the hemicellulose that surrounds it. Hemicelluloses are diverse and amorphous, with β-1,4-xylan being the primary constituent. Therefore, xylanase, an enzyme that exhibits both endo- and exo-activity, is a crucial component for breaking down hemicellulose.

In this example, an enzyme complex called Viscozyme and Pectinex, produced by Novozymes in Bagsvaerd, Denmark, was employed for the enzymatic pretreatment and extraction of yerba mate leaves.

Viscozyme, derived from Aspergillus aculeatus, contains a combination of enzymes, including cellulase, hemicellulase, arabanase, beta-glucanase, and xylanase. This enzyme complex is known to have a declared activity level of 100 FBG/g (FBG=Fungal Beta-Glucanase unit), making it suitable for breaking down the complex cell wall structures and improving the extraction of bioactive compounds from yerba mate leaves. Pectinex 5XL was derived from Aspergillus niger, having activity level of 5000 ferment depectinization units (FDU)/ml.

TABLE 2
Effect of enzyme and combinations on the processing yield of bioactive compounds

| | | | Bioactive purity % | | | |
|---|---|---|---|---|---|---|
| | | | (Bioactive processing yield %) | | | |
| | | | | | Caffeoyl- | IC50 DPPH |
| Sample | | Yield | | | quinic | antioxidant, |
| Number | Solvent | % | Theobromine | Caffeine | acid | ug/mL |
| PL20230197 | Water (control) | 19.00 | 0.59 | 2.19 | 26.16 | 456 |
| | | | (0.11) | (0.42) | (4.97) | |
| PL20230212 | 0.1% Viscozyme | 48.61 | 0.46 | 1.68 | 16.91 | 387 |
| | | | (0.22) | (0.82) | (9.40) | |
| PL20230213 | 1% Viscozyme | 56.86 | 0.47 | 1.69 | 16.10 | 355 |
| | | | (0.27) | (0.96) | (8.36) | |
| PL20230214 | 2% Viscozyme | 54.85 | 0.47 | 1.79 | 17.13 | 358 |
| | | | (0.26) | (0.98) | (9.40) | |
| PLS20230217 | 0.1% Viscozyme + | 57.6 | 0.44 | 1.62 | 18.2 | 335 |
| | 0.1% Pectinex | | (0.25) | (0.93) | (10.47) | |
| PLS20230218 | 1% Viscozyme + | 55.0 | 0.44 | 1.57 | 15.6 | 359 |
| | 1% Pectinex | | (0.23) | (0.90) | (8.69) | |
| PLS20230219 | 2% Viscozyme + | 54.3 | 0.42 | 1.64 | 15.81 | 360 |
| | 2% Pectinex | | (0.23) | (0.89) | (8.58) | |

Table 2 shows the effect of using the enzyme Viscozyme to extract compounds from yerba mate leaves. Viscozyme produced around 2-3 times more total extract (48.0-56.0 g per 100 g dry weight) than water (19 g per 100 g dry weight). Viscozyme was also more effective at extracting specific compounds from yerba mate leaves. For example, using water only, 0.1 g theobromine, 0.4 g caffeine, and 4.97 g caffeolyquinic acids were extracted from 100 g dry yerba mate leaves. However, using 0.1% Viscozyme, 0.22 g theobromine, 0.82 g caffeine and 9.40 g caffeoylquinic acids were extracted from 100 g dry yerba mate leaves. Further increasing the dosage of Viscozyme to 1% and 2% only slightly improved the extraction yield. Overall, Viscozyme was found to be an effective enzyme for extracting compounds from yerba mate leaves. A dosage of 0.1% Viscozyme was found to be sufficient to achieve good extraction yields.

Pectin is a crucial component in plant cell walls, playing a major role as an adhesive between cells. Pectinex, which is derived from Aspergillus aculeatus, is an enzyme complex containing a variety of enzymes, including pectinase, cellulase, β-galactosidase, polygalacturonase, and fructosyltransferase, with polygalacturonase being the most prominent enzyme in the mix. This enzyme complex is particularly effective at breaking down pectin. Therefore, in this example, Pectinex was introduced as a second enzyme to further enhance the processing efficiency of yerba mate leaves. The data indicates that the addition of 0.1% Pectinex in the system increased the yield percentage from 48% to 57%. This suggests that the application of enzymes like Pectinex and Viscozyme significantly improves processing yield. These enzymes, specifically pectinases and carbohydrases from Pectinex and Viscozyme, were found to degrade pectin and cellulose in cell walls.

Consequently, the release of cell contents increases the yield of yerba mate. Generally, a concentration of 0.2% showed the highest yield, with no significant differences between samples treated with 0.2% and 4% enzyme concentration. Therefore, 0.2% (w/w) was considered the optimal concentration for enhancing yerba mate processing yield. Among the three studied enzymes, the combination of Pectinex Ultra SP-L and Viscozyme demonstrated a more positive effect than their individual use, indicating that a diverse enzyme mixture could be more efficient in treating yerba mate leaves.

In addition to evaluating extraction yields, this example also examined the levels of caffeine, theobromine, total caffeoylquinic acid content, and DPPH scavenging activity in both untreated and treated samples, as presented in Table 2. Overall, there was an increasing trend in the yield of bioactive compounds as the enzyme dosage increased, with the highest values observed in the sample treated with 0.1% Viscozyme and 0.1% Pectinex. These results indicate that enzyme treatment can indeed enhance the extraction of phenolic compounds, in line with recent research suggesting that enzyme addition facilitates the release of potentially cell wall-bound phenolics.

However, importantly the content of bioactive compounds in the treated samples was lower compared to that in the untreated sample. Without being bound by theory, this phenomenon may possible be attributed to the enzyme-catalyzed degradation of high-molecular-weight substances within the plant cell wall matrix. This process leads to the release of more water and other soluble solids rather than phenolic compounds. While this increased the overall extraction yield, it resulted in a reduction in the concentration of bioactive compounds. The complexity of plant cell walls and the specific locations of bioactive compounds, as well as their bonding, which can influence extraction, remain largely unknown. Additionally, the concentration or content of phenolics in the treated extracts is also influenced by the extraction of intracellular water, further contributing to this observed phenomenon.

Table 2 also shows that the antioxidant activity of the control sample was measured at 456 μg/ml. However, enzymatic treatment significantly enhances this antioxidant activity, especially in terms of DPPH scavenging activity. Importantly, the change in DPPH scavenging activity does not necessarily follow the same pattern as the total phenolic content. For instance, in the samples treated with enzymes, DPPH activity increased while the total phenolic acid content decreased.

Without being bound by theory, this divergence may possibly be explained by the presence of various components in yerba mate leaves beyond phenolic compounds that contribute to the observed antioxidant activity. These include substances like vitamin C, gallic acid, chlorophyll, or specific poly/oligosaccharides. Collectively, these compounds likely play a role in enhancing the overall antioxidant activity, even when changes in total phenolic content suggest otherwise.

TABLE 3
Influence of incubation time on yerba mate extraction
using 0.1% Viscozyme and 0.1% Pectinex

| | | Content | | | Yield | | |
|---|---|---|---|---|---|---|---|
| | | | | Caffeoyl- | | | Caffeoyl- |
| Time | Yield | Theobromine | Caffeine | quinic | Theobromine | Caffeine | quinic |
| (hr) | (%) | (%) | (%) | acid (%) | (%) | (%) | acid (%) |
| 1 | 44.9 | 0.53 | 1.83 | 19.39 | 0.24 | 0.82 | 8.71 |
| 2 | 49.13 | 0.43 | 1.55 | 16.66 | 0.21 | 0.76 | 8.18 |
| 3 | 47.68 | 0.50 | 1.75 | 18.74 | 0.24 | 0.84 | 8.93 |
| 4 | 48.98 | 0.45 | 1.67 | 17.70 | 0.22 | 0.82 | 8.67 |
| 16 | 52.25 | 0.5 | 1.6 | 16.9 | 0.24 | 0.86 | 8.85 |
| 20 | 53.54 | 0.43 | 1.53 | 15.72 | 0.23 | 0.82 | 8.42 |

Table 3 illustrates the processing yield achieved over a 20-hour incubation period using an optimized enzyme combination (0.1% Viscozyme+0.1% Pectinex). Enzymatic treatment significantly improved processing efficiency, resulting in a 2-3-fold increase compared to the control. While there is an initial increase in processing yield within the first hour, there are no significant changes observed between 2 hours and 20 hours of incubation.

The data suggests that extending the treatment duration can enhance the yield, but this effect was mainly observed during the first hour of reaction. The increased yield over time is possibly attributed to the greater breakdown of cell walls, releasing soluble compounds. Notably, the combination of Pectinex and Viscozyme enzymes outperformed their individual counterparts, resulting in an approximately 8% increase in yield.

Considering the data on yield, total phenolic content, it can be concluded that a 2-hour incubation duration is suitable for these enzymatic treatments. While longer durations may provide some additional benefits, the differences are not significant, making shorter processing times more efficient.

TABLE 4
Effect of incubation temperature on yerba mate extraction

| | Temperature | | |
|---|---|---|---|
| | 40° C. | 50° C. | 60° C. |
| Processing yield | 45.92 | 44.72 | 45.38 |

CONTENT

| | | | |
|---|---|---|---|
| Theobromine | 0.47 | 0.48 | 0.49 |
| 5-caffeoylquinic acid | 6.28 | 6.37 | 6.60 |
| Chlorogenic acid | 3.19 | 3.30 | 3.52 |
| Caffeine | 1.67 | 1.70 | 1.76 |
| 4-caffeoylquinic acid | 1.64 | 1.80 | 1.88 |
| Rutin | 0.25 | 0.27 | 0.26 |
| 3,5-dicaffeoyl-quinic acid | 6.41 | 6.45 | 5.27 |
| 4,5-dicaffeoyl-quinic acid | 0.85 | 0.85 | 0.77 |
| Total caffeoyl-quinic acid | 18.36 | 18.77 | 18.05 |

YIELD

| | | | |
|---|---|---|---|
| Theobromine | 0.22 | 0.21 | 0.22 |
| 5-caffeoylquinic acid | 2.88 | 2.85 | 3.00 |
| Chlorogenic acid | 1.46 | 1.48 | 1.60 |
| Caffeine | 0.77 | 0.76 | 0.80 |
| 4-caffeoylquinic acid | 0.75 | 0.81 | 0.85 |
| Rutin | 0.11 | 0.12 | 0.12 |
| 3,5-dicaffeoyl-quinic acid | 2.94 | 2.88 | 2.39 |
| 4,5-dicaffeoyl-quinic acid | 0.39 | 0.38 | 0.35 |
| Total caffeoyl-quinic acid | 8.43 | 8.39 | 8.19 |

Table 4 shows that temperature had a relatively minor impact in the range of 40-60° C., with higher temperatures resulting in a slight decrease in caffeoylquinic acid content. This can likely be attributed to several factors related to temperature's influence on the extraction process.

However, as the temperature increased, the viscosity of the solution decreased. This decrease in viscosity possibly improved the penetration of enzymes into the cell walls, allowing them to more effectively degrade the substrates, such as pectin and cellulose. This enzymatic action facilitated the release of phenolic compounds stored within the vacuoles of the plant cells. Secondly, higher temperatures caused a reduction in surface tension, which had a softening effect on the cell walls. This softening weakened the structural integrity of the cell walls, making it easier for phenolic compounds to be released. However, the higher temperature could have led to the degradation of polyphenols, resulting in a reduction in caffeoylquinic acids. Secondly, the elevated temperature might have caused the denaturation of enzymes, diminishing their effectiveness in releasing phenolic compounds from the plant cells.

In summary, temperature played a role in enhancing the extraction of phenolic compounds from yerba mate leaves by affecting factors such as solution viscosity, cell wall softening, and enzyme activity. However, at the highest temperature, some degradation of polyphenols and enzyme denaturation may have led to a decrease in caffeoylquinic acid content. The optimum condition is 50° C.

### Example 4: Effect of pH and Buffer

Enzymes are highly sensitive to the pH level of their surrounding environment. These proteins have specific active sites that enable them to catalyze chemical reactions, and the ionizable amino acid residues within these active sites can exist in various ionic forms depending on the pH of the solution. As a result, the pH of the reaction medium plays a pivotal role in determining the efficiency of enzyme activity.

In this example, two multi-enzyme complexes were used. Viscozyme and Pectinex, both of which contain a mix of enzymes, including cellulase, hemicellulase, arabanase, beta-glucanase, and xylanase.

Importantly, each of these enzymes may have its own optimal pH range for activity, where it performs most effectively. To control the pH of the reaction medium, citric acid/sodium citrate buffer was utilized. A range of pH values, spanning from 3 to 7, were systematically explored to assess their impact on the extraction of bioactive compounds from yerba mate. The results, as presented in Table 5, demonstrated that pH levels falling within the range of 5.9 to 6.2 were found to be the most conducive for extracting bioactive compounds from yerba mate. It is likely that these pH values align with the optimal pH conditions for the enzymes present in Viscozyme and Pectinex, as evidenced by the higher total phenolic content obtained within this pH range. However, straying from this optimal pH range, either towards more acidic or more alkaline conditions, was found to have a detrimental effect on the extraction of bioactives. Particularly, lower pH levels resulted in a reduction in the total phenolic content, suggesting that the enzymes within Viscozyme and Pectinex were less active under acidic conditions.

In summary, pH and buffer solutions were found to exert a profound influence on enzyme-assisted extraction processes. The pH level of the extraction medium is important as it can impact both enzyme activity and the solubility of the target compounds. For example, cellulase enzymes tend to work best in a slightly acidic to neutral pH range, whereas pectinase enzymes may favor a more acidic environment.

Moreover, pH can also influence the solubility of bioactive compounds. Some compounds are more soluble at specific pH levels. By adjusting the pH to the optimal range for the target compounds, the efficiency of their extraction may possibly be enhanced. For example, alkaline conditions may improve the solubility of phenolic compounds, while certain alkaloids like caffeine may be more soluble in acidic conditions.

TABLE 5
Effect of buffer pH on yerba mate extraction

| | Solvent | | | | | | |
|---|---|---|---|---|---|---|---|
| | | | 0.1 mM | 1 mM | 25 mM | 50 mM | 100 mM |
| | | 50 nm | citric | citric | citrate | citrate | citrate |
| | Water | HCl | acid | acid | buffer | buffer | buffer |
| pH | 6.1 | 4.7 | 4.1 | 3.2 | 6.2 | 6.1 | 5.9 |
| Yield | 19.00 | 44.72 | 31.59 | 29.30 | 39.54 | 44.66 | 60.45 |

PURITY %

| | | | | | | | |
|---|---|---|---|---|---|---|---|
| Theobromine | 0.59 | 0.48 | 0.78 | 0.70 | 0.61 | 0.50 | 0.39 |
| 5-caffeoylquinic acid | 7.23 | 6.37 | 8.88 | 9.11 | 7.72 | 5.85 | 4.67 |
| Chlorogenic acid | 4.54 | 3.30 | 5.28 | 5.19 | 4.41 | 3.33 | 2.60 |
| Caffeine | 2.19 | 1.70 | 2.79 | 2.68 | 2.17 | 1.82 | 1.40 |
| 4-caffeoylquinic acid | 2.47 | 1.80 | 4.29 | 3.73 | 2.85 | 2.64 | 1.98 |
| Rutin | 0.31 | 0.27 | 0.44 | 0.38 | 0.29 | 0.22 | 0.17 |
| 3,5-dicaffeoyl-quinic | 10.34 | 6.45 | 12.75 | 11.22 | 12.01 | 9.04 | 7.35 |
| acid | | | | | | | |
| 4,5-dicaffeoyl-quinic | 1.58 | 0.85 | 3.23 | 2.20 | 2.01 | 2.12 | 1.53 |
| acid | | | | | | | |
| Total caffeoyl-quinic | 26.16 | 18.77 | 34.44 | 31.45 | 29.00 | 22.98 | 18.14 |
| acid | | | | | | | |

YIELD %

| | | | | | | | |
|---|---|---|---|---|---|---|---|
| Theobromine | 0.11 | 0.21 | 0.25 | 0.21 | 0.24 | 0.22 | 0.23 |
| 5-caffeoylquinic acid | 1.37 | 2.85 | 2.81 | 2.67 | 3.05 | 2.61 | 2.82 |
| Chlorogenic acid | 0.86 | 1.48 | 1.67 | 1.52 | 1.75 | 1.49 | 1.57 |
| Caffeine | 0.42 | 0.76 | 0.88 | 0.79 | 0.86 | 0.81 | 0.85 |
| 4-caffeoylquinic acid | 0.47 | 0.81 | 1.36 | 1.09 | 1.13 | 1.18 | 1.20 |
| Rutin | 0.06 | 0.12 | 0.14 | 0.11 | 0.12 | 0.10 | 0.10 |
| 3,5-dicaffeoyl-quinic | 1.96 | 2.88 | 4.03 | 3.29 | 4.75 | 4.04 | 4.45 |
| acid | | | | | | | |
| 4,5-dicaffeoyl-quinic | 0.30 | 0.38 | 1.02 | 0.64 | 0.79 | 0.95 | 0.93 |
| acid | | | | | | | |
| Total caffeoyl-quinic | 4.97 | 8.39 | 10.88 | 9.22 | 11.47 | 10.26 | 10.97 |
| acid | | | | | | | |

Phosphate buffers are commonly employed in protein purification and chromatography processes due to their reduced likelihood of denaturing proteins. These buffers are also favored in many biological assays because they are less likely to disrupt the reactions. Phosphate buffers offer several advantages, including a broader pH range (from 5.8 to 8.0, as opposed to the narrower range of 3.0 to 6.2 found in some other buffers). They exhibit a higher buffering capacity, are less acidic, and remain more stable. Furthermore, phosphate salts are readily available and cost-effective, making them a practical and economical choice for buffer solutions.

TABLE 6
Yerba Mate processing yield by using KH₂PO₄ as buffer by
using different concentration of enzyme from 0.2% to 2%

| Sample | | | | Stage 1 | Stage 2 | Stage 1 + 2 |
|---|---|---|---|---|---|---|
| number | Solvent | KH₂PO₄ | Enzyme | yield | yield | total yield |
| PLS20230549 | water | | | 19.1% | 6.8% | 25.8% |
| PLS20230544 | water | 0.05M | 0.20% | 26.7% | 9.9% | 36.6% |
| PLS20230545 | water | 0.05M | 0.50% | 28.5% | 7.8% | 36.3% |
| PLS20220548 | water | 0.05M | 2% | 32.9% | 10.3% | 43.2% |
| PLS20220546 | water | 0.075M, 1% | 0.50% | 32.7% | 9.5% | 42.2% |
| PLS20220547 | water | 0.1M | 0.50% | 34.7% | 9.0% | 43.6% |

TABLE 7
The purity of bioactives in different yerba mate extracts

| | | | | | | | | | Total |
|---|---|---|---|---|---|---|---|---|---|
| | | | | Chloro- | | | | | caffeoyl- |
| | | | | genic | | | 3,5-di- | 4,5-di- | quinic |
| | Theobromine | Caffeine | 5-CQA | acid | 4-CQA | Rutin | CQA | CQA | acid |
| commercial | 0.79% | 3.80% | 8.33% | 5.22% | 5.76% | 1.22% | 9.96% | 7.34% | 36.60% |
| extracts | | | | | | | | | |
| PLS20230549 | 1.09% | 4.22% | 12.71% | 6.21% | 4.05% | 1.07% | 15.98% | 4.05% | 43.00% |
| PLS20230544 | 0.95% | 3.67% | 10.88% | 5.36% | 3.58% | 0.94% | 13.47% | 3.40% | 36.70% |
| PLS20230545 | 0.96% | 3.52% | 10.89% | 5.31% | 3.25% | 0.84% | 14.61% | 2.99% | 37.04% |
| PLS20220548 | 0.86% | 3.08% | 10.03% | 4.76% | 2.81% | 0.81% | 13.86% | 2.78% | 34.25% |
| PLS20230546 | 0.81% | 3.03% | 9.54% | 4.72% | 2.58% | 0.77% | 12.26% | 2.50% | 31.59% |
| PLS20230547 | 0.77% | 2.87% | 9.82% | 4.67% | 2.70% | 0.86% | 11.94% | 2.05% | 31.19% |

As shown in Tables 6 and 7, Enzyme-assisted extraction significantly increased the processing yield from 25% to over 40% when compared to water extraction. This increase was influenced by the strength of the buffer and the quantity of enzyme used.

A higher extraction yield, while beneficial in terms of quantity, often results in lower purity of bioactive compounds. This occurs because a larger number of phytonutrients are extracted, which dilutes the concentration of the specific bioactive compound. For instance, in the case of caffeine, a water extraction method yielded 25%, with a purity of 4.22% and a yield of 1.092%. In contrast, when the extraction yield increased to 43.2% (as observed in PLS20230548), the purity of caffeine decreased to approximately 3.08%. However, the yield increased to 1.254%, representing a 22% increase. Enzyme-assisted extraction improved the total extraction yield of caffeoylquinic acid by about 33%, primarily due to the weakly acidic pH of the buffer, which favors the extraction of chlorogenic acids over caffeine.

Based on this example, buffer conditions can be used to control the trade-off between the yield and purity of yerba mate extracts. Opting for conditions that maximize extraction yield will typically result in lower purity. Conversely, choosing conditions that minimize extraction yield, such as in water extraction, will yield extracts with the highest purity for all bioactive compounds.

Buffer solutions are useful when the pH of the starting material or reaction mixture needs adjustment. Since enzymes are sensitive to pH fluctuations, buffer solutions play a role in stabilizing and sustaining a constant pH, which may prevent adverse impacts on enzymatic reactions. By using buffer solutions, the pH may be set to a preferred range for both enzyme activity and the solubility of the target compounds. This ensures that the extraction process operates at peak efficiency and consistency.

TABLE 8
Physical properties of yerba mate spray-dried powder and
spray-drying efficiency with different feed properties

| | Total | | | | | | | |
|---|---|---|---|---|---|---|---|---|
| | solids | Ratio of | Moisture | Bulk | Tapped | | | Recovery |
| | content | extract to | content | density | density | Compressibility | Hausner | yield |
| | (% w/w) | Fibersol-2 | (% w/w) | (g/mL) | (g/mL) | index | ratio | (%) |
| 1 | 1.5 | 0 | 5.37 | 0.48 | 0.77 | 17.98 | 1.12 | 63.93 |
| 2 | 5 | 0 | 4.88 | 0.43 | 0.68 | 16.80 | 1.18 | 64.33 |
| 3 | 7.5 | 0 | 4.13 | 0.43 | 0.61 | 19.59 | 1.12 | 72.09 |
| 4 | 10 | 0 | 4.21 | 0.45 | 0.62 | 14.56 | 1.11 | 75.64 |
| 5 | 15 | 0 | 4.26 | 0.47 | 0.77 | 19.34 | 1.15 | 81.64 |
| 6 | 15 | 3:1 | 3.93 | 0.50 | 0.76 | 10.56 | 1.13 | 90.0 |

Table 8 summarizes the characteristics of the yerba mate spray dried powders. The moisture content of the yerba mate spray dried powder was measured to be in the range of 4.13% to 5.37%. No noticeable difference was observed among the samples, thus no relationship between total solids content powder moisture content was observed. However, powders started to cake and form lumps after 1 day of storage open packaging. Hence, in some instances, spray dried yerba mate extracts may need to be stored in sealed package to prevent caking. While the moisture content of the powder was not compromised, feed extract of higher solids content can improve the cost-efficiency of spray drying process.

The mean size of the particles was estimated to be in the range of 5-25 μm, based on the configuration of spray dryer in this example. It can be categorized as a microfine powder, according to definition in British Pharmacopoeia for particles with not less than 90% by weight passes through sieve of aperture size 45 μm.

The bulk densities of the yerba mate spray dried powder ranged from 0.40 to 0.50 g/ml.

Flow character is rated based on Compressibility Index (CI) and Hausner Ratio (HR) (Carr, 1965; Hausner, 1967). Lower CI or lower Hausner ratios of a material indicate better flow properties than higher ones. A Carr's CI of <10 or HR of <1.11 is considered ‘excellent’ flow whereas CI>38 or HR>1.60 is considered ‘very poor’ flow. There are intermediate scales for CI between 11-15 or HR between 1.12-1.18 is considered ‘good’ flow, CI between 16-20 or HR between 1.19-1.25 is considered ‘fair’ flow, CI between 21-25 or HR between 1.26-1.34 is considered passable flow, CI between 26-31 or HR between 1.35-1.45 is considered ‘poor’ flow, and CI between 32-37 or HR between 1.46-1.59 is considered ‘very poor’ flow A higher Hausner Ratio and Carr Index or Compressibility Index (CI) indicates poorer powder flowability. HR value larger than 1.29 and CI value larger than 26 indicate the samples are of poor flow property whereas powder of excellent flow has an HR value at 5 to 10 and CI value at 1 to 1.18. In this example, the CI and HR value showed that the yerba mate spray dried powder has good flow properties. The HR and CI value obtained were found not to vary according to the changes in total solids content of the extracts.

The spray drying process yielded improved results when using a 3:1 ratio of extract solids to Fibersol-2. Without being bound by theory, this effect can possibly be attributed to reduced wall deposition during the process.

### Example 5:100 kg Batch Yerba Mate Leaf Cut Enzymatic Extraction

Preparing the Buffer Solution: A 25 mM trisodium citrate/citric acid buffer solution was created by adding 5.68 kg of Trisodium Citrate (molecular weight: 258.06 g/mol) and 0.62 kg of Citric Acid (molecular weight: 192.124 g/mol) to 750 L of water. Water was gradually added until the total volume reached 1000 L.

Loading the Extractor: 100 kg of yerba mate leaf cut and 1000 L of the buffer solution was placed into the extractor. The contents were thoroughly mixed until the temperature reaches 55° C.

Adding Enzyme Solution: 200 grams of a pre-mixed enzyme solution was introduced into the extractor. The contents were vigorously mixed for 2 hours at 55° C.

Inactivating the Enzyme Activity: The mixture was heated to approximately 95° C. The temperature was maintained for 30 minutes to deactivate the enzyme. Separating the Liquid Fraction 1: A 5 micro filter was used to separate the liquid fraction 1 from the remaining yerba mate leaf residues.

Preparing 800 L 1 mM Citric Acid Water Solution: A 1 mM citric acid solution was created by adding 153.6 grams of Citric Acid (molecular weight: 192.124 g/mol) to 600 L of water. Water was gradually added until the total volume reached 800 L.

Performing Extraction with 1 mM Citric Acid in Water: 800 L of the 1 mM Citric Acid Water solution was added to the yerba mate leaf residues. The extraction process was conducted at a temperature of 70° C. for 2 hours.

Separating the Liquid Fraction 2: A 5 micro filter was used to separate the liquid fraction 2 from the yerba mate leaf residues.

Combining the Liquid Fractions: Liquid fractions 1 and 2 are merged together. A vacuum was used to concentrate the combined solution to a concentration of greater than 13.5% within a temperature range of 60° C.-70° C.

Resistant maltodextrin was added in a ratio of 1:3 to the extracts.

Spray Drying: The concentration solution was spray dried. The inlet temperature was set to 160° C.-165° C. and the outlet temperature was set to 90° C.-95° C.

### Example 6: Improving Health and Well Being

The following example demonstrates the improvement in health and wellbeing by ingesting the compositions and formulations described herein.

A plant extract is extracted using the methods described herein, including the methods described in Examples 1 and 2. A composition is formulated from the plant extract, the composition including the plant extract obtained by using enzyme as processing aid, and/or hydrolyzed protein powder. The composition is formulated as a powder or concentrated fluid. The composition is included in a drink, such as a tea, and ingested. Ingesting the composition results in improved health and well-being, as determined by improved blood lipid levels, reduced body weight, reduced fasting blood sugar levels, improved gut health, improved heart health, increased feelings of positive experiences, and decreased feelings of negative experiences.

### Example 7: Saponin Enzyme Assisted Extraction

The following example relates to saponin testing to assess the potential benefits of this compound.

Yerba mate (Ilex paraguariensis A. St. Hil.) was collected from the São Mateus do Sul region in the State of Paraná, Brazil. The leaves and branches underwent traditional processing for enzymatic inactivation using a method known as “sapeco.” In this process, the yerba mate was initially exposed to a temperature of 400° C., which was then reduced to 65° C., with an average processing time of 8 seconds. Subsequently, the yerba mate was dried in a dryer at temperatures ranging from 90 to 110° C. for approximately 3 hours. Once dried, the leaves were coarsely ground to facilitate handling, resulting in what is commonly referred to as “canchada” yerba mate. In the present example, canchada yerba mate was used as raw material.

Saponins are plant-based compounds known for their foaming properties. Traditionally used in soaps, saponins are now valued in the food industry for their potential health benefits. While commonly found in plant roots, research has shown that saponins can also be present in plant leaves, such as tea leaves. In this example, the saponin content of yerba mate extracts was analyzed to evaluate its potential health benefits.

Five grams of dried extract was dissolved in 15 ml of high-purity water. The solution was filtered to remove any insoluble particles. The filtrate was diluted to a final volume of 100 milliliters with the same solvent. Fifteen milliliters of hydrochloric acid was added to achieve a concentration of 4 ml/L. The mixture was heated under reflux for 2 hours to break down the saponin molecules. The sapogenins were extracted from the acidic solution using chloroform. This extraction process was repeated four times to ensure complete recovery. The combined chloroform extracts were evaporated to dryness to obtain the sapogenins. The residue was dissolved in acetonitrile and filtered through a 0.45-micron membrane to remove any impurities. The final solution was analyzed using high-performance liquid chromatography (HPLC). This process was repeated three times for reproducibility.

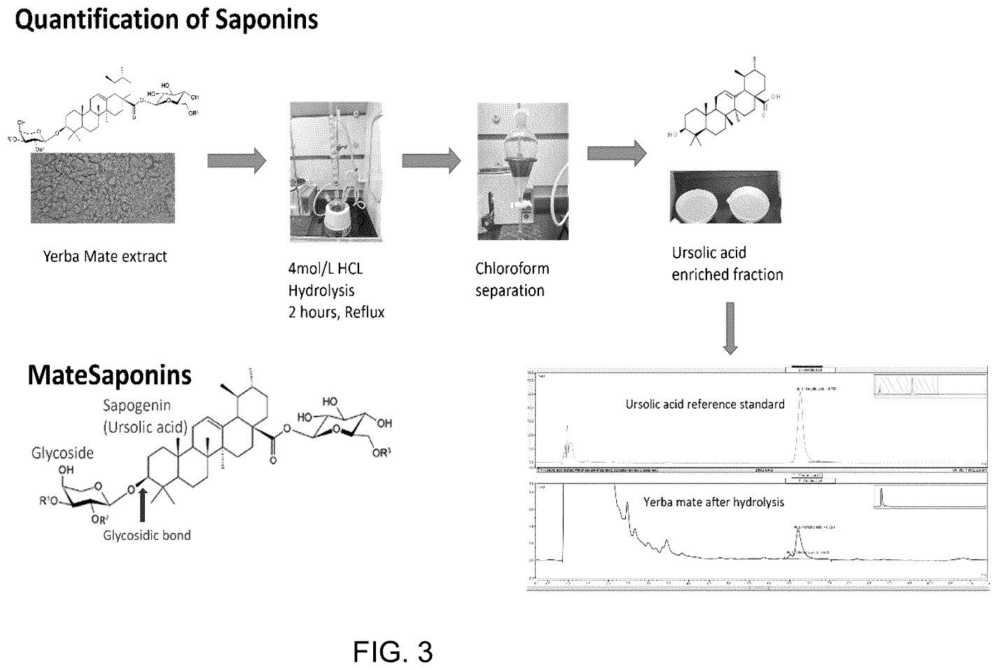

HPLC analyses were performed using a Thermo Fisher (Carlsbad, CA) Vanquish series HPLC system, including Chromeleon software, a VC-P10-A-01 pump, and a VC-D11-A-01 diode array detector (DAD) set at 203 nanometers. Ursolic acid was analyzed using a reversed-phase Accucore C18 column (150 mm×2.1 mm, particle size 2.6 μm, Thermo Fisher). The mobile phase consisted of a mixture of acetonitrile and water (70:30, volume/volume). The mobile phase was degassed using an ultrasound bath and filtered under vacuum through a membrane. The flow rate was set at 0.4 ml/min. The HPLC analysis was conducted at room temperature. Additional information about the HPLC assay method can be found in Gnoatto et al., J. Braz. Chem. Soc. 16 (4), August 2005, doi.org/10.1590/S0103-50532005000500007. FIG. 3 illustrates the experimental procedures used in this example.

Total saponin content (TSC) of Yerba mate extract was analyzed using vanillin-sulfuric acid assay. Briefly, 8% (w/v) vanillin in ethanol (100 μL) and 72% (v/v) sulfuric acid (1 mL) were added to the plate containing sample or oleanolic acid (100 μL), kept at 60° C. for 10 min in the water bath, and then cooled on ice for 5 min. After that, the plate was measured at 544 nm using a microplate reader. The TSC is displayed as mg oleanolic acid equivalent (OAE) per gram of Yerba mate dry weight (mg OAE/g DW). Additional information about the vanillin-sulfuric acid assay method can be found at Le et al., Technologies 2018, 6 (3), 84; doi.org/10.3390/technologies6030084.

The saponins in Yerba mate are ursolic and oleanolic acid aglycons, which contribute to its flavor and biological activities, including anti-inflammatory, hypocholesterolemic, and antiparasitic effects.

Yerba Mate Canchada was processed by different enzyme, the details samples information and testing data are shown in Table 9 and Table 10:

TABLE 9

| Sample Number | Enzyme |
|---|---|
| PLS20240413 | water |
| PLS20240390 | Pectinex & Viscozyme mixture/ 0.5%, pH 5-6 |
| PLS20240391 | Viscozyme/ 1% |
| PLS20240392 | Depol 283L/ 1% |
| PLS20240393 | Viscozyme & Depol 283L/ 2% |
| PLS20240394 | Pectinex & Viscozyme mixture/ 0.5% pH 4.5 |

TABLE 10

| | PLS20240412 | PLS20240390 | PLS20240491 | PLS20240392 | PLS20240393 | PLS20240394 |
|---|---|---|---|---|---|---|
| Theobromine | 0.51% | 0.21% | 0.25% | 0.26% | 0.16% | 0.22% |
| Caffeine | 2.96% | 4.58% | 4.30% | 3.39% | 3.27% | 3.06% |
| 5-caffeoylqinic acid | 7.07% | 7.06% | 8.87% | 8.24% | 7.86% | 7.98% |
| Chlorogenic acid | 7.49% | 6.63% | 7.05% | 6.90% | 7.15% | 5.42% |
| 4-caffeoylquinic acid | 7.45% | 6.02% | 7.00% | 6.73% | 6.35% | 3.50% |
| Rutin | 1.99% | 1.96% | 2.22% | 2.06% | 1.89% | 2.18% |
| 3.4-dicaffeoylquinic | 2.69% | 4.39% | 3.65% | 3.60% | 3.31% | 1.24% |
| acid | | | | | | |
| 3,5-dicaffeoylquinic | 2.66% | 6.75% | 6.08% | 5.44% | 5.07% | 4.70% |
| acid | | | | | | |
| 4,5-dicaffeoylquinic | 3.37% | 4.89% | 4.48% | 4.07% | 3.66% | 1.85% |
| acid | | | | | | |
| Total caffeoylquinic | 30.73% | 35.74% | 37.13% | 34.98% | 33.40% | 24.69% |
| acids | | | | | | |
| Total Saponin Content | 4.27% | 10.06% | 6.42% | 3.81% | 6.03% | 4.86% |
| DPPH | 178.65 | 116.40 | 135.14 | 208.52 | 131.61 | 208.16 |

The water extraction without enzymes yielded 4.27% saponin content, which serves as a baseline. Pectinex & Viscozyme mixture (0.5%) resulted in the highest saponin content at 10.1%, more than doubling the water extraction yield. Viscozyme (1%) alone increased saponin content to 6.4%. Viscozyme & Depol 283L (2%) increased saponin content to 6.0%. Depol 283L (1%) decreased saponin content to 3.8% compared to water extraction. Pectinex & Viscozyme mixture (0.5%, PH 4-5) yielded 4.9% saponin content.

The Pectinex & Viscozyme mixture at pH 5-6 yields the highest saponin content. This may be due to synergistic action of these enzymes in breaking down plant cell walls. Pectinase, found in Pectinex, breaks down pectin, a key component of plant cell walls. This process helps release saponins trapped within the cell structure by degrading the jelly-like matrix that holds plant cells together. Viscozyme contains a range of enzymes that further break down complex carbohydrates in the cell walls, enhancing the release of saponins and other compounds. The combination of these enzymes is particularly effective because they target different components of the cell wall, leading to more efficient breakdown and release of saponins compared to using each enzyme individually.

On the contrary, Depol 283L yielded the lowest saponin content, which may be due to several factors. Depol 283L may not effectively target the specific components of the plant cell walls that contain saponins. If the enzyme does not efficiently break down these structures, fewer saponins are released. The activity of Depol 283L might not be optimal under the conditions used, such as pH or temperature. Enzymes have specific conditions where they function best, and any deviation can reduce their effectiveness. It is possible that Depol 283L could degrade saponins or interfere with their extraction by breaking down other compounds that stabilize saponins within the plant matrix.

In addition, by using Pectinex and Viscozyme mixture, pH 5-6 is double efficient for extraction compared to pH 4-5. This can be attributed to several factors. Enzymes like pectinase and those in Viscozyme have optimal pH ranges where they are most active. Pectinase typically works best between pH 3.0 and 6.54. Within this range, the enzymes can more effectively break down plant cell walls, facilitating the release of saponins. At a slightly higher pH (5-6), the cell wall components may become more permeable, allowing enzymes to penetrate and act more efficiently on the cell wall matrix, thus releasing more saponins. The solubility of saponins and the stability of the extracted compounds might be enhanced at this pH range, improving extraction efficiency. Higher pH levels can reduce protein content that might otherwise bind or trap saponins within the plant matrix. This reduction can lead to a more efficient release of saponins.

Overall, the Pectinex & Viscozyme mixture maximizes saponin extraction by effectively breaking down various structural components of the plant cells.

Interestingly, there appears to be an inverse relationship between saponin content and antioxidant activity as measured by DPPH. The treatment with the highest saponin content (Pectinex & Viscozyme mixture, 10.1%) showed the lowest DPPH value (116.4). Conversely, the treatment with the lowest saponin content (Depol 283L, 3.8%) exhibited the highest DPPH value (208.52).

This observation is supported by the calculated Pearson correlation coefficient between saponin content and DPPH values, which is approximately-0.82. This strong negative correlation indicates that as saponin content increases, the DPPH value tends to decrease. Lower DPPH values typically indicate higher antioxidant activity. This is because DPPH is a stable free radical, and antioxidants neutralize it, causing a decrease in absorbance. Therefore, the Pectinex & Viscozyme mixture treatment, having the highest saponin content, showed the highest antioxidant activity. The Depol 283L treatment, with the lowest saponin content, demonstrated the lowest antioxidant activity.

Different enzyme treatments appear to have distinct effects on both saponin content and antioxidant activity.

Viscozyme-containing mixtures (Pectinex & Viscozyme) seem to enhance saponin extraction while also increasing antioxidant activity. Viscozyme alone (1%) results in moderate saponin content and antioxidant activity. Depol 283L (1%) appears to be less effective for saponin extraction but may preserve or enhance other antioxidant compounds.

The findings of this example suggest an inverse relationship between saponin content and antioxidant activity as measured by DPPH. Enzyme treatments with higher saponin content (e.g., Pectinex & Viscozyme mixture) exhibited lower DPPH values, indicating higher antioxidant activity. Conversely, treatments with lower saponin content (e.g., Depol 283L) displayed higher DPPH values, suggesting lower antioxidant activity. This correlation was supported by a strong negative Pearson correlation coefficient of approximately-0.82.

The choice of enzyme treatment significantly impacted both saponin extraction and antioxidant activity. Viscozyme-containing mixtures were particularly effective in extracting saponins and enhancing antioxidant properties. While Viscozyme alone produced moderate results, Depol 283L was less efficient in extracting saponins but may have preserved or promoted other antioxidant compounds.

### Example 8: Glucosidase Inhibitory Activity Assay

In the following example, yerba mate extract and fractions produced using different enzyme at different conditions as described in Example 7 were evaluated for their inhibitory effect on α-glucosidase enzymes by in-vitro method.

The α-glucosidase inhibitory activity assay is a key method used to assess potential treatments for type 2 diabetes and related metabolic disorders. This assay measures how effectively a substance can inhibit the enzyme α-glucosidase, which plays a crucial role in breaking down complex carbohydrates into simple sugars, primarily glucose, in the small intestine. By inhibiting α-glucosidase, the digestion of carbohydrates is slowed, leading to delayed and reduced glucose absorption into the bloodstream. This helps manage postprandial (after-meal) blood glucose levels, which is vital for individuals with type 2 diabetes or impaired glucose tolerance.

Substances that demonstrate strong α-glucosidase inhibitory activity have potential as treatments for type 2 diabetes. They can help reduce hyperglycemia (high blood sugar) by slowing carbohydrate digestion and glucose absorption. Plant-based compounds are being explored as potential α-glucosidase inhibitors, as these natural alternatives may offer fewer side effects compared to synthetic drugs like acarbose, miglitol, and voglibose, commonly used α-glucosidase inhibitors that can cause gastrointestinal discomfort.

In this Example, yerba mate extracts were evaluated for their α-glucosidase inhibitory activity by using epigallocatechin gallate (EGCG) as a positive control. EGCG is a well-known natural compound found in green tea with demonstrated α-glucosidase inhibitory properties. The yerba mate extracts were compared to EGCG to assess the relative effectiveness of these extracts in inhibiting α-glucosidase, to determine if yerba mate extracts have potential as natural alternatives for managing blood glucose levels in individuals with type 2 diabetes or related metabolic disorders.

An assay to measure α-glucosidase inhibitory activity of extract and fractions was carried out according to the standard method with minor modification. In a 96-well plate, reaction mixture containing 50 μl phosphate buffer (100 mM, pH=6. 8), 10 μl alpha-glucosidase (1 U/ml), and 20 μl of varying concentrations of extract and fractions (0.1, 0.2, 0.3, 0.4, and 0.5 mg/ml) was preincubated at 37° C. for 15 min. Then, 20 μl P-NPG (5 mM) were added as a substrate and incubated further at 37° C. for 20 min. The reaction was stopped by adding 50 μl Na₂ CO₃ (0.1 M). The absorbance of the released p-nitrophenol was measured at 405 nm using Multiplate Reader. Acarbose at various concentrations (0.1-0.5 mg/ml) was included as a standard. Without test substance was set up in parallel as a control and each experiment was performed in triplicates. The results were expressed as percentage inhibition, which was calculated using the formula,

Inhibitory activity (%)=(1−As/Ac)×100
where, As is the absorbance in the presence of test substance and Ac is the absorbance of control.

In the present Example, 5 yerba mate extracts produced by using different enzyme at different conditions as described in Example 7 were evaluated for their inhibitory effect on α-glucosidase enzymes by in-vitro method. The extraction methods were those described in Example 7 above, see Table 9.

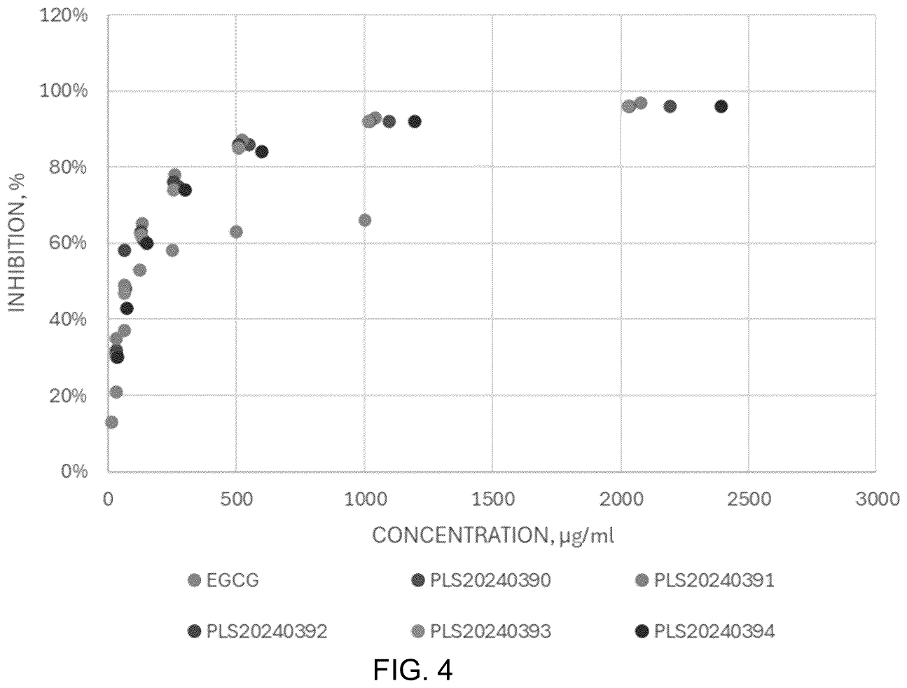

FIG. 4 illustrates the percent inhibition of α-glucosidase inhibition by Yerba mate extracts and EGCG, and Table 11 shows the α-glucosidase inhibitory effects.

TABLE 11

| | IC50 values of |
|---|---|
| Samples | α-glucosidase, μg/ml |
| EGCG | 138.6 |
| PLS20240390 | 184.24 |
| PLS20240391 | 171.47 |
| PLS20240392 | 165.73 |
| PLS20240393 | 150.85 |
| PLS20240394 | 184.4 |

EGCG was used as a standard reference, which showed α-glucosidase inhibitory activity with an IC50 value of 138 μg/ml. Among all, PLS20240393 (Viscozyme & Depol 283L/2%) showed the best α-glucosidase inhibitory activity with an IC50 value 150 μg/ml, which is comparable to EGCG.

The glucosidase inhibitory assay values range from 150.85 to 184.4 across the samples. This assay measures the ability of compounds to inhibit α-glucosidase, an enzyme involved in carbohydrate digestion, which is relevant for managing blood sugar levels in conditions like diabetes.

The correlation analysis shows how each compound correlates with glucosidase inhibition. Caffeine has a positive correlation (r=0.636) meaning higher caffeine content is associated with stronger glucosidase inhibition. Rutin (r=0.480), Theobromine (r=0.398), and Total Saponin Content (r=0.370) also show positive correlations, suggesting they may contribute to glucosidase inhibition.

On the other hand, several caffeoylquinic acids show negative correlations, such as:

Chlorogenic acid (r=−0.871) has a strong negative correlation, indicating that higher chlorogenic acid content is associated with lower glucosidase inhibitory activity. Other caffeoylquinic acids like 4-caffeoylquinic acid (r=−0.553) and Total Caffeoylquinic Acids (r=−0.581) also negatively correlate with glucosidase inhibition.

The mean values of the compounds across all samples provide a general overview of their typical concentrations: Caffeine: 3.92% Total Caffeoylquinic Acids: 32.19% DPPH: 159.97 μg/ml (indicating antioxidant activity) Total Saponin Content: 6.24% Glucosidase Inhibitory Assay: 171.34 μg/ml.

Compounds like caffeine, rutin, and theobromine seem to enhance α-glucosidase inhibition, which could make them useful in developing treatments for managing blood sugar levels. The negative correlations observed for chlorogenic acid and other caffeoylquinic acids suggest that these compounds might counteract or reduce the inhibitory effect on α-glucosidase.

The data suggests that certain compounds, particularly caffeine, rutin, and saponin, positively influence glucosidase inhibitory activity, while others like chlorogenic acid may reduce this effect. This balance between different compounds could be important when considering these samples for potential therapeutic use in managing blood sugar levels or diabetes-related conditions.

What is claimed is:

1. A composition, comprising:
an enzyme;
a plant extract obtained from a plant or a hydrolyzed protein powder,
wherein the composition is formulated as a powder or a concentrated fluid.

2. The composition of claim 1, wherein the enzyme is present in an amount of about 0.01% to about 5% w/w, and wherein the enzyme is configured as a processing aid.

3. The composition of claim 1, wherein the enzyme is a cellulase, hemicellulase, pectinase, α-amylase, β-amylase, xylanase, β-glucanase, protease, phytase, esterase, endo-β-1,4-glucanase, cellobiohydrolase, proteinase, or a combination thereof.

4. The composition of claim 1, further comprising one or more endo-β-glucanases or one or more polygalacturonases.

5. The composition of claim 1, wherein the plant is yerba mate or cocoa.

6. The composition of claim 1, wherein the hydrolyzed protein powder is beef protein, whey protein, soy protein, pea protein, or rice protein.

7. The composition of claim 1, wherein the plant extract is caffeine, theobromine, caffeoylquinic acid, rutin, triterpenoid, or chlorogenic acid, or a combination thereof.

8. A method for extracting a plant extract from a plant material or for producing a hydrolyzed protein, the method comprising:
providing a buffer solution;
mixing the plant material or a protein with the buffer solution to form a homogenized slurry;
adding an enzyme to the homogenized slurry to form a slurry-enzyme mixture;
heating the slurry-enzyme mixture to a first temperature;
maintaining the slurry-enzyme mixture at the first temperature;
heating the slurry-enzyme mixture to a second temperature;
maintaining the slurry-enzyme mixture at the second temperature; and
separating the slurry-enzyme mixture into a solid fraction and a liquid fraction, wherein the liquid fraction comprises the plant extract or the hydrolyzed protein.

9. The method of claim 8, wherein the buffer solution comprises citric acid, tricalcium citrate, or sodium hydroxide.

10. The method of claim 8, wherein the buffer solution comprises potassium dihydrogen phosphate or dipotassium hydrogen phosphate.

11. The method of claim 8, wherein the pH of the buffer solution is between about 5.0 and about 6.2.

12. The method of claim 8, wherein the first temperature is in a range from about 40° C. to about 60° C.

13. The method of claim 8, wherein the second temperature is in a range from about 90° C. to about 99° C.

14. The method of claim 8, wherein the plant material comprises yerba mate leaves or cocoa pod husk, or wherein the protein comprises beef protein.

15. The method of claim 8, wherein the plant extract is caffeine, theobromine, rutin, chlorogenic acid, an isomer of caffeoylquinic acids (CQA), polyphenols, saponin, or a combination thereof.

16. The method of claim 8, wherein the enzyme is a cellulase, hemicellulase, pectinase, α-amylase, β-amylase, xylanase, β-glucanase, protease, phytase, esterase, endo-β-1,4-glucanase, cellobiohydrolase, proteinase, or a combination thereof.

17. The method of claim 8, further comprising spray-drying the plant extract with a solution comprising resistant maltodextrin (RMD).

18. A method of extracting a plant extract from plant material comprising:
providing plant material;
treating said plant material with one or more enzymes comprising a pectinase, a hemicellulase, a beta-glucanase, or a xylanase; and
extracting the plant extract from the treated plant material, wherein the extraction is conducted at a pH of 5-6.

19. The method of claim 18, wherein the plant material is yerba mate.

20. The method of claim 18, wherein the plant extract is a saponin.
